(19) United States
(12) Patent Application Publication
(10) Pub. No.: US 2007/0219448 A1
(43) Pub. Date: Sep. 20, 2007

(54) Method and Apparatus for Selective Treatment of Tissue

(75) Inventors: Ralf Seip, Indianapolis, IN (US); Wo-Hsing Chen, Fishers, IN (US); Roy F. Carlson, New Palestine, IN (US); Narendra T. Sanghv, Indianapolis, IN (US); Kris A. Dines, Indianapolis, IN (US); Michael A. Penna, Indianapolis, IN (US); Richard Pfile, Indianapolis, IN (US)

Correspondence Address: BAKER & DANIELS LLP, 300 NORTH MERIDIAN STREET, SUITE 2700, INDIANAPOLIS, IN, 46204, US

(73) Assignee: FOCUS SURGERY, INC., Indianapolis, IN (US)

(21) Appl. No.: 11/568,599

(22) PCT Filed: May 5, 2005
(86) PCT No.: PCT/US05/15648

§ 371 Date: Nov. 28, 2006

Related U.S. Application Data
(60) Provisional application No. 60/568,556, filed on May 6, 2004.

(51) Int. Cl. A61B 8/06

(52) U.S. Cl. 600/454; 600/437; 600/453

(57) ABSTRACT

A IIIFU System (100) is disclosed which may automatically generate a proposed treatment plan for treating a tissue treatment area (10) with HIFU Therapy. In one example, the proposed treatment plan includes a plurality of treatment sites selected based on a three-dimensional model generated from ultrasound data. In another example, the proposed treatment plan excludes portions of the tissue treatment area (10) corresponding to blood flow, such as the neuro-vascular bundles (20) when treating the prostate (11).

### RELATED APPLICATIONS

This application claims the benefit of U.S. Provisional Patent Application Ser. No. 60/568,556, filed May 6, 2004, titled TREATMENT OF SPATIALLY ORIENTED DISEASE WITH A SINGLE THERAPY, IMAGING AND DOPPLER ULTRASOUND TRANSDUCER, the disclosure of which is expressly incorporated by reference herein.

### NOTICE

This invention was made with government support under grant reference number 5R44DK059664-03 awarded by National Institutes of Health (NIH). The Government has certain rights in the invention.

### BACKGROUND OF THE INVENTION

The present invention relates ultrasound systems and in particular to high intensity focused ultrasound (“HIFU”) systems and the treatment of tissue with HIFU Systems. The treatment of tissue with high intensity focused ultrasound (“HIFU”) energy is known in the art. For instance, HIFU may be used in the treatment of benign prostatic hyperplasia (BPH) and prostate cancer (PC). Further, it is known to use Doppler imaging to locate portions of tissue to be treated with HIFU energy.

HIFU Systems are known for the treatment of diseased tissue. An exemplary HIFU system is the Sonablate®-500 HIFU system available from Focus Surgery located at 3940 Pendleton Way, Indianapolis, Ind. 46226. The Sonablate® 500 HIFU system uses a dual-element, confocal ultrasound transducer which is moved by mechanical methods, such as motors, under the control of a controller. Typically one element of the transducer is used for imaging and the other element of the transducer is used for providing HIFU Therapy. A typical treatment procedure for treating the prostate with the Sonablate® 500 HIFU system includes using the imaging element of the transducer to create both two dimensional sector (or transverse) and two dimensional linear (or sagittal) ultrasound scans of the prostate capsule, manually defining treatment zones in multiple sector images (treatment sites placed in defined treatment zone by system), and using the therapy element of the transducer to provide HIFU Therapy to the patient. The treated site is then imaged to determine the effects of the HIFU Therapy. The positioning of the transducer, provision of HIFU Therapy, and post-imaging steps are repeated for each particular portion of tissue which is to be treated. All of these steps take place while the patient is immobilized on a treatment table.

The Sonablate® 500 HIFU system is particularly designed to provide HIFU Therapy to the prostate. However, as stated in U.S. Pat. No. 5,762,066, the disclosure of which is expressly incorporated by reference herein, the Sonablate® 500 HIFU system and/or its predecessors may be configured to treat additional types of tissue.

Further details of suitable HIFU systems may be found in U.S. Pat. No. 5,762,066; U.S. Abandoned patent application Ser. No. 07/840,502 filed Feb. 21, 1992, Australian Patent No. 5,732,801; Canadian Patent No. 1,332,441; Canadian Patent No. 2,250,081; U.S. Pat. No. 5,036,855; U.S. Pat. No. 5,117,832; U.S. Pat. No. 5,492,126; U.S. Pat. No. 6,685,640, the disclosures of which are expressly incorporated by reference herein.

### SUMMARY OF THE INVENTION

As used herein the term “HIFU Therapy” is defined as the provision of high intensity focused ultrasound to a portion of tissue at or proximate to a focus of a transducer. It should be understood that the transducer may have multiple foci and that HIFU Therapy is not limited to a single focus transducer, a single transducer type, or a single ultrasound frequency. As used herein the term “HIFU System” is defined as a system that is at least capable of providing a HIFU Therapy.

In an exemplary embodiment of the present invention, a method of providing treatment to a tissue treatment area including a plurality of tissue components is provided. The method comprising the steps of: generating ultrasound data related to the tissue treatment area; and automatically generating a proposed treatment plan of the tissue treatment area. The proposed treatment plan including a plurality of treatment sites selected to receive HIFU Therapy. The plurality of treatment sites being selected based on a three-dimensional model of a first tissue component located in the tissue treatment area. The three-dimensional model of the first tissue component being based on the generated ultrasound data In one example, the method further comprises the steps of: detecting blood flow in the tissue treatment area; and excluding a first portion of tissue from the proposed treatment plan, the exclusion of the first portion of tissue being based on the detection of blood flow at a location generally corresponding to the first portion of tissue. In one exemplary refinement, the exclusion of the first portion of tissue is further based on the location of the first portion relative to the three-dimensional model of the first tissue component. In another exemplary refinement, the tissue treatment area generally corresponds to a prostate of a patient, the first tissue component corresponds to a prostatic capsule, and the first portion generally corresponds to a neuro-vascular bundles and wherein the step of generating the ultrasound data includes the steps of positioning an ultrasound transducer proximate to the tissue treatment area by the transrectal insertion of the ultrasound transducer and obtaining multiple two-dimensional images of the tissue treatment area including a plurality of sector images and a plurality of linear images. In yet another example, the three-dimensional model is generated by the steps of: locating a first boundary trace of the first tissue component in a first set of ultrasound data generally corresponding to a first plane; locating a second boundary trace of the first tissue component in a second set of ultrasound data generally corresponding to a second plane, the second plane being generally orthogonal to the first plane; and computing a boundary surface of the first tissue component based on the first boundary trace and the second boundary trace. In still a further example, the method of claim 1, further comprises the steps of: presenting the proposed treatment plan on a display device along with a three dimensional representation of the tissue treatment area for review by a user; determining a first location within the tissue treatment area having blood flow associated therewith; further presenting on the display for review by a user an indicia to indicate the presence of blood flow at the first location, the indicia being positioned to correspond to the first location; receiving a modification to the proposed treatment plan from the user thereby generating a modified proposed treatment plan; and commencing the modified proposed treatment.

In another exemplary embodiment of the present invention, a method of providing treatment to a tissue treatment area including a plurality of tissue components is provided. The method comprising the steps of: generating ultrasound data related to the tissue treatment area; generating blood flow data related to the tissue treatment area; determining the location of a first tissue component based on the ultrasound data; determining the location of a second tissue component based on the blood flow data; and automatically generating a proposed treatment plan of the tissue treatment area, the proposed treatment plan including a plurality of treatment sites, the plurality of treatment sites being selected such that HIFU Therapy is provided to the first tissue component and such that the second tissue component is excluded from HIFU Therapy. In one example, the blood flow data is generated by Doppler ultrasound imaging and wherein the location of the first tissue component is determined based on a three-dimensional model of the first tissue component, the three-dimensional model of the first tissue component being based on the ultrasound data. In one exemplary refinement, the location of the second tissue component is determined based on an indication of the presence of blood flow at the location of the second tissue component and the relative position of the of the location of the second tissue component and the three-dimensional model of the first tissue component. In still a further exemplary refinement, the method further comprises the steps of: presenting on a display device for review by a user; a three dimensional representation of the tissue treatment area; a plurality of treatment indicia, each of the treatment indicia corresponding to a respective treatment site, a representation of a boundary of the first tissue component, the boundary being determined from the three-dimensional model of the first tissue component; and a blood flow indicia indicating the location of the second tissue component, the blood flow indicia providing an indication of the amount of blood flow; receiving a modification to the proposed treatment plan from the user thereby generating a modified proposed treatment plan; and commencing the modified proposed treatment plan. In another example, the tissue treatment area generally corresponds to a prostate of a patient, the first tissue component generally corresponds to a prostatic capsule, and the second tissue component generally corresponds to a neuro-vascular bundles and wherein the step of generating the ultrasound data includes the steps of positioning an ultrasound transducer proximate to the tissue treatment area by the transrectal insertion of the transducer and obtaining multiple two-dimensional images of the tissue treatment area including a plurality of sector images and a plurality of linear images.

In a further exemplary embodiment of the present invention, an apparatus for treating a tissue treatment area including a plurality of tissue components. The apparatus comprising: a transducer which is positionable proximate to the tissue treatment area of tissue, the transducer being configured to emit ultrasound energy and to sense ultrasound energy; and a controller operably coupled to the transducer. The controller being configured to operate the transducer in an imaging mode wherein images of the tissue are obtained by ultrasound energy sensed by the transducer and blood flow information and in a therapy mode wherein portions of the tissue in the tissue treatment area are treated with a HIFU Therapy with the transducer. The controller being further configured to automatically generate a proposed treatment plan having a plurality of treatment sites, the plurality of treatment sites of the proposed treatment plan being selected to provide HIFU Therapy to a first tissue component, while excluding a second tissue component from HIFU Therapy, the location of the second tissue component being determined based on blood flow information obtained during the imaging mode of operation. In one example, the tissue treatment area generally corresponds to a prostate of a patient and wherein the transducer is contained within a probe, the probe being configured for transrectal insertion to position the transducer proximate to the tissue treatment area. In one exemplary refinement, the first tissue component generally corresponds to a prostatic capsule, and the second tissue component generally corresponds to a neuro-vascular bundles. In another exemplary refinement, the location of the first tissue component is determined based on a three-dimensional model of the first tissue component. The three-dimensional model being generated by the steps of: locating a first boundary trace of the first tissue component in a first image obtained during the imaging mode of operation, the first image generally corresponding to a first plane; locating a second boundary trace of the first tissue component in a second image obtained during the imaging mode of operation, the second image generally corresponding to a second plane, the second plane being generally orthogonal to the first plane; and computing a boundary surface of the first tissue component based on the first boundary trace and the second boundary trace. In still a further example, the apparatus further comprises a display device and the controller being configured to present with the display device for review by a user a three dimensional representation of the tissue treatment area, a plurality of treatment indicia, each of the treatment indicia corresponding to a respective treatment site in the proposed treatment plan, a representation of a boundary of the first tissue component, the boundary being determined from a three-dimensional model of the first tissue component; and a blood flow indicia indicating the location of the second tissue component. In one exemplary refinement, the apparatus further comprises an user input device and the controller being configured to generate a modified proposed treatment plan based on a requested modification received with the user input device.

In still a further exemplary embodiment of the present invention, an apparatus for treating a tissue treatment area including a plurality of tissue components is provided. The apparatus comprising: a transducer which is positionable proximate to the tissue treatment area, the transducer being configured to emit ultrasound energy and to sense ultrasound energy; and a controller operably coupled to the transducer. The controller being configured to operate the transducer in an imaging mode wherein images of the tissue are obtained by ultrasound energy sensed by the transducer and in a therapy mode wherein portions of the tissue in the tissue treatment area of tissue are treated with a HIFU therapy with the transducer. The controller being further configured to automatically generate a proposed treatment plan of the tissue treatment area, the proposed treatment plan including a plurality of treatment sites selected based on a three-dimensional model of a first tissue component located in the tissue treatment area of tissue. The three-dimensional model of the first tissue component being based on the images obtained in the imaging mode of operation. In one example, the tissue treatment area generally corresponds to a prostate of a patient and wherein the transducer is contained within a probe, the probe being configured for transrectal insertion to position the transducer proximate to the tissue treatment area. In another example, the three-dimensional model of the first tissue component is generated by the steps of: locating a first boundary trace of the first tissue component in a first image obtained during the imaging mode of operation, the first image generally corresponding to a first plane; locating a second boundary trace of the first tissue component in a second image obtained during the imaging mode of operation, the second image generally corresponding to a second plane, the second plane being generally orthogonal to the first plane; and computing a boundary surface of the first tissue component based on the first boundary trace and the second boundary trace. In a further example, the apparatus further comprises a display device and the controller being configured to detect the presence of blood flow in the tissue treatment area of tissue and present with the display device for review by a user a three dimensional representation of the tissue treatment area including the three-dimensional model of the first tissue component, a plurality of treatment indicia, each of the treatment indicia corresponding to a respective treatment site in the proposed treatment plan, and a blood flow indicia indicating the location of blood flow in the tissue treatment area. In still a further example, the apparatus further comprises an user input device and the controller being configured generate a modified proposed treatment plan based on a requested modification received with the user input device.

In yet another exemplary embodiment of the present invention, a method for treating tissue in a tissue treatment area including tissue components is provided. The method comprising the steps of: providing a HIFU system having software configured to provide therapy to diseased tissue by focusing ultrasound proximate to the diseased tissue and further configured to provide location information of blood flow in the tissue and location information on at least one of the tissue components; identifying potential treatment areas based on the location information of the tissue components; excluding potential treatment areas based on the location information of blood flow in the tissue treatment area; and providing therapy to all of the identified non-excluded treatment areas with the HIFU system In one example, the location information of the tissue components is presented to the a user by the following steps: identifying the location of blood flow with Doppler imaging; identifying the location of at least some of the tissue components with at least one of ultrasound 2-D imaging and ultrasound 3-D imaging; generating a three-dimensional model representation of the location of tissue components based on the location of blood flow and the ultrasound imaging information; and displaying the three-dimensional model on a display.

In yet a further exemplary embodiment of the present invention a method of providing treatment to a tissue treatment area including a prostatic capsule and a neuro-vascular bundles is provided. The method comprising the steps of: imaging the tissue treatment area; and automatically generating a proposed treatment plan of the tissue treatment area, the proposed treatment plan including a plurality of treatment sites selected to receive HIFU Therapy, the plurality of treatment sites being selected to provide HIFU Therapy to the prostatic capsule and to exclude the neuro-vascular bundles from the provision of HIFU Therapy. In one example, the location of the neuro-vascular bundles is determined based on blood flow information obtained during the imaging of the tissue treatment area. In yet another example, the method further comprises the steps of: presenting the proposed treatment plan to a user for review; receiving a modification to the proposed treatment plan from the user; and generating a modified proposed treatment plan based on the received modification.

Additional features of the present invention will become apparent to those skilled in the art upon consideration of the following detailed description of the illustrative embodiment exemplifying the best mode of carrying out the invention as presently perceived.

### BRIEF DESCRIPTION OF THE DRAWINGS

The detailed description of the drawings particularly refers to the accompanying figures in which:

FIG. 1 is a representative view of an exemplary HIFU System including a transducer which is to be positioned proximate to a tissue treatment area;

FIG. 2 is an isometric view of an exemplary embodiment of the HIFU System of FIG. 1 including an enlarged view of a probe tip, the probe tip being illustrated with various sector image planes that come to be imaged with a transducer at the probe;

FIG. 2A is a representation of a probe of the HIFU System of FIG. 2 illustrating two types of images which are to be obtained during an imaging of the tissue treatment area;

FIG. 3 illustrates an exemplary embodiment of the controller of FIG. 1;

FIG. 4A is a representative view of a first exemplary PW Doppler imaging system including multiple gates;

FIG. 4B is a representative view of a first exemplary CFI Doppler imaging system including multiple gates;

FIG. 4C is a representative view of a second exemplary CFI Doppler imaging system including multiple gates in software;

FIG. 5 illustrates an exemplary method of operation of the controller of FIG. 1;

FIG. 6A illustrates another exemplary method of operation of the controller of FIG. 1;

FIG. 6B is a representation of an automatic treatment planning module of FIG. 6A;

FIG. 7 illustrates an exemplary method of the automatic treatment planning module of FIG. 6B;

FIG. 8A illustrates an exemplary tracing/marking user interface to be shown with the display device of the system of FIG. 1 for indicating the location of various tissue components in the tissue treatment area;

FIG. 8B illustrates an exemplary screen shot of the display device of the system of FIG. 1 showing multiple sector and linear image views of the tissue treatment area to be potentially selected for tracing/marking the tissue components;

FIG. 9 illustrates exemplary shape models;

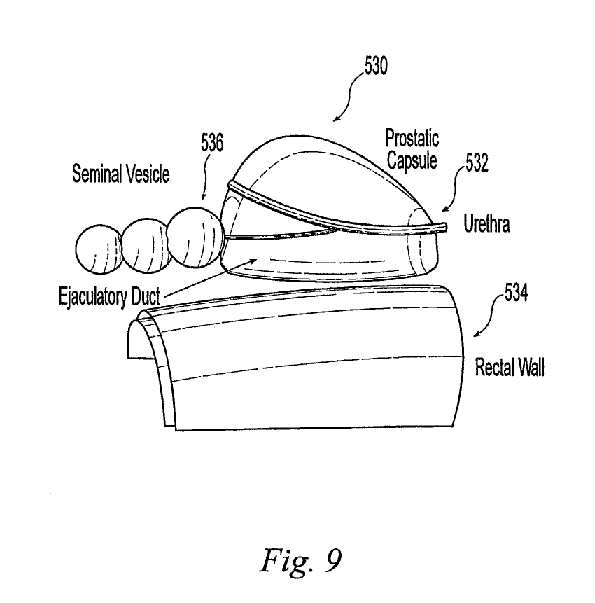

FIG. 10A illustrates an exemplary sector view for display with the display device of the system of FIG. 1, including indicia of proposed treatment sites of the proposed treatment plan and indicia or representations indicating sites or regions of blood flow;

FIG. 10B illustrates exemplary sector view for display with the display device of the system of FIG. 1, including indicia of proposed treatment sites of the proposed treatment plan;

FIG. 11 illustrates a plurality of representative sector views for display with the display device of the system of FIG. 1, each of the sector views to contain indicia of proposed treatment sites of the proposed treatment plan;

FIG. 12 illustrates a volume image of the tissue treatment area for display with the display device of the system of FIG. 1, the volume image containing indicia of proposed treatment sites of the proposed treatment plan;

FIG. 13 illustrates a volume image of the tissue treatment area for display with the display device of the system of FIG. 1, the volume image containing indicia of proposed treatment sites of the proposed treatment plan and shape models of the various tissue components, and indicia or representations indicating sites or regions of blood flow;

FIG. 14 is an exemplary representation of an exemplary lesion library; and

FIG. 15 illustrates several exemplary lesions of varying sizes from the lesion library of FIG. 14.

### DETAILED DESCRIPTION OF THE DRAWINGS

The present application is directed to the treatment of diseases of the prostate with a HIFU system. However, it should be understood that the HIFU system may be implemented to treat other diseased tissues located at other regions in the body.

As described more fully herein, in one embodiment, the apparatus and methods of the present application generate an automatic treatment plan for treatment of a tissue treatment area containing the diseased tissue. The automatic treatment plan is tailored to treat the diseased tissue and to selectively exclude the treatment of portions of the tissue treatment area. In one embodiment, the portions of the tissue treatment area selected for exclusion in the treatment plan are selected based on the location of one or more tissue components, the detection of blood flow, and/or physician input. In the case of treating the prostate, the portions of the tissue treatment area selected for exclusion are selected to minimize side effects of the treatment such as impotency, erectile dysfunction and/or incontinence.

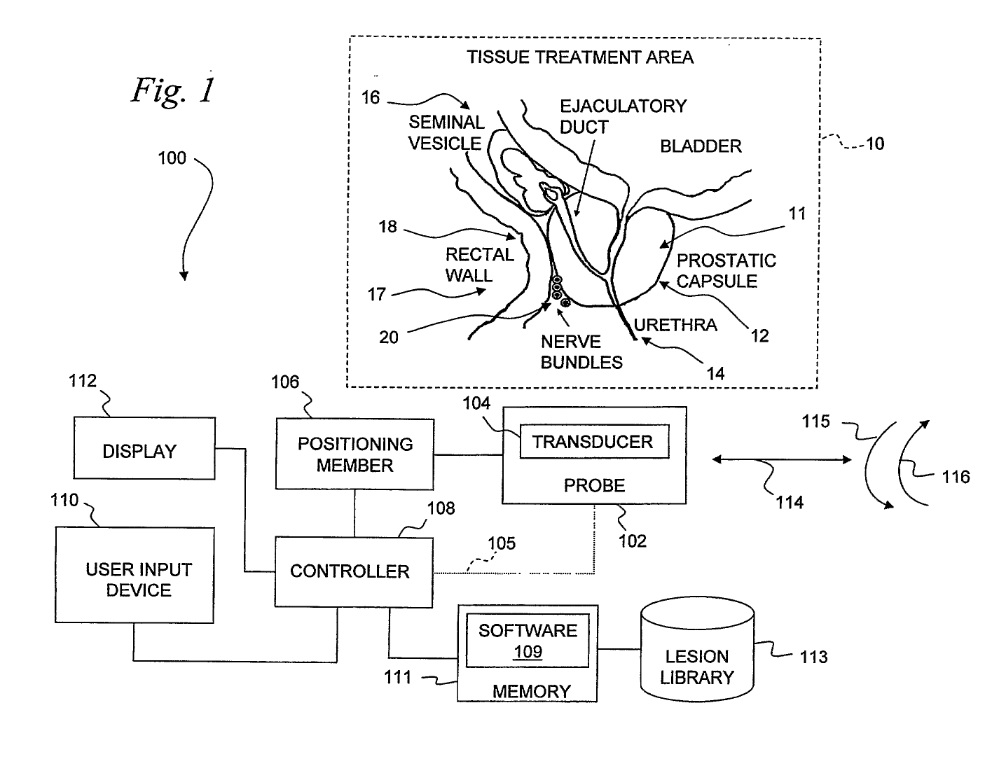

Referring to FIG. 1, several anatomical structures and their locations that are relevant to HIFU treatment planning for treatment of the prostate are shown. Referring to FIG. 1, a tissue treatment area or region 10 is shown. Tissue treatment area 10 is illustratively shown to include the following tissue components: a prostate 11 having a prostatic capsule 12, urethra 14, seminal vesicles 16, and a rectum 17 having a rectal wall 18. Further, neuro-vascular bundles (“NVB”) 20 are shown wrapping tightly around the periphery of prostatic capsule 12. The NVB's are critical in the ability of the patient to achieve erections. As such, damage to NVB 20 may result in the patient experiencing impotence, erectile dysfunction and incontinence.

The location of the NVB 20 can be determined based on the locations of the vascular component of the NVB 20. The vascular component of the NVB 20, if healthy, includes blood flowing through respective blood vessels. Such blood flow may be detected with Doppler ultrasound imaging. Doppler ultrasound imaging techniques are well known in the art, Doppler ultrasound imaging may also be used to detect early stage prostate cancer in the patient due to the fact that early stage prostate cancer forms neo-vascularization.

An exemplary HIFU system 100 is shown in FIG. 1. HIFU system 100 includes a probe 102 having a transducer member 104, a positioning member 106, a controller 108 operably coupled to probe 102 and the positioning member 106, a user input device 110 (such as keyboard, trackball, mouse, and/or touch screen), and a display 112. Probe 102 is operably connected to controller 108 through positioning member 106. However, as indicated by line 105 probe 102 may be directly connected with controller 108. Positioning member 106 is configured to linearly position transducer member 104 along line 114 and to angularly position transducer member 104 in directions 115, 116.

In one exemplary embodiment, controller 108 includes software 109 located in memory 111. Software 109 controls the operation of HIFU System 100 including the imaging of the tissue treatment area 10, the automatic planning of a proposed HIFU treatment, and the provision of HIFU Therapy during treatment.

Transducer member 104 is positioned generally proximate to a region of tissue treatment area 10. In the case of the prostate, transducer 104 is positioned generally proximate to the prostate by the transrectal insertion of probe 102. Transducer member 104 is moved by positioning member 106 and controlled by controller 108 to provide imaging of at least a portion of the tissue in tissue treatment area 10 and to provide HIFU therapy to portions of the tissue within tissue treatment area 10. In one embodiment, prostatic capsule 12, urethra 14, seminal vesicles 16, rectal wall 18, are NVB 20 are included in the portions of tissue imaged. As such, HIFU system 100 may operate in an imaging mode wherein at least a portion of the tissue within tissue treatment area 10 may be imaged and in a therapy mode wherein HIFU therapy is provided to portions of the tissue within tissue treatment area 10.

In one embodiment, transducer member 104 is a single crystal two element transducer. A central element is used for imaging and a surrounding element is used for HIFU Therapy. In one embodiment, both elements work at 4 MHz. In another embodiment, the HIFU Therapy element operates at 4 MHz and the imaging element operates at 5-7 MHz. An exemplary transducer is disclosed in U.S. Pat. No. 5,117,832, the disclosure of which is expressly incorporated herein by reference. However, one skilled in the art will appreciate that various transducer configurations may be implemented. In a one embodiment, transducer 104 is capable of providing imaging of at least a portion of the tissue within tissue treatment area 10 and of providing HIFU therapy to at least a portion of the tissue within tissue treatment area 10.

However, the present invention is not limited to the type of transducer implemented. On the contrary, various transducer geometries having a single focus or multiple foci and associated controls may be used including transducers which are phased arrays, such as the transducers disclosed in pending U.S. patent application Ser. No. 11/070,371, filed on Mar. 2, 2005 (“'371 Application”), the disclosure of which is expressly incorporated herein by reference. As explained in the '371 Application, at least one transducer disclosed therein has a scanning aperture. As such, the disclosed transducer does not require positioning member 106 to translate the disclosed transducer in direction 114 during imaging and treatment.

Additional exemplary transducers and associated controls are disclosed in U.S. Pat. No. 5,762,066; U.S. Abandoned patent application Ser. No. 07/840,502 filed Feb. 21, 1992; Australian Patent No. 5,732,801; Canadian Patent No. 1,332,441; Canadian Patent No. 2,250,081; U.S. Pat. No. 5,036,855; U.S. Pat. No. 5,492,126; U.S. Pat. No. 6,685,640, each of which is expressly incorporated herein by reference. In one embodiment, a phased array transducer, Model No. 8EC4 available from Terason® located at 77-79 Terrace Hill Ave., Burlington, Mass. 01803 is incorporated into probe 102.

In a preferred embodiment, transducer 104 is capable of providing imaging information about the tissue in tissue treatment area 10 (such as a plurality of two-dimensional images), to provide HIFU therapy to at least a portion of the tissue in the tissue treatment area 10, and to provide Doppler imaging of at least a portion of the tissue in the tissue treatment area 10. In one embodiment, transducer 104 is a single transducer having at least two transducer elements. In another embodiment, transducer 104 is comprised of multiple transducers, each having one or more elements.

Probe 102 is configured to be positioned next to the rectal wall 18 of a patient and to be fixably secured relative to the patient during a treatment procedure as described below. By fixing the location of probe 102 relative to the patient it is possible to repeatably locate transducer member 104 relative to the patient with positioning member 106. Such repeatability is important due to requirements of the treatment procedure to first determine the location of various tissue components within the tissue treatment area 10 with ultrasound imaging, the determination of potential treatment zones or treatment sites based on the identified location information, and the subsequent placement of transducer member 104 to provide HIFU Therapy to locations in the tissue treatment area 10 corresponding to the treatment zones or treatment sites. Additional details of suitable ultrasound systems and methods of using high intensity focused ultrasound to treat tissue are disclosed in U.S. Pat. No. 5,762,066; U.S. Abandoned patent application Ser. No. 07/840,502 filed Feb. 21, 1992, Australian Patent No. 5,732,801; Canadian Patent No. 1,332,441; Canadian Patent No. 2,250,081; U.S. Pat. No. 5,036,855; U.S. Pat. No. 5,117,832; U.S. Pat. No. 5,492,126; U.S. Pat. No. 6,685,640, the disclosures of which are expressly incorporated by reference herein.

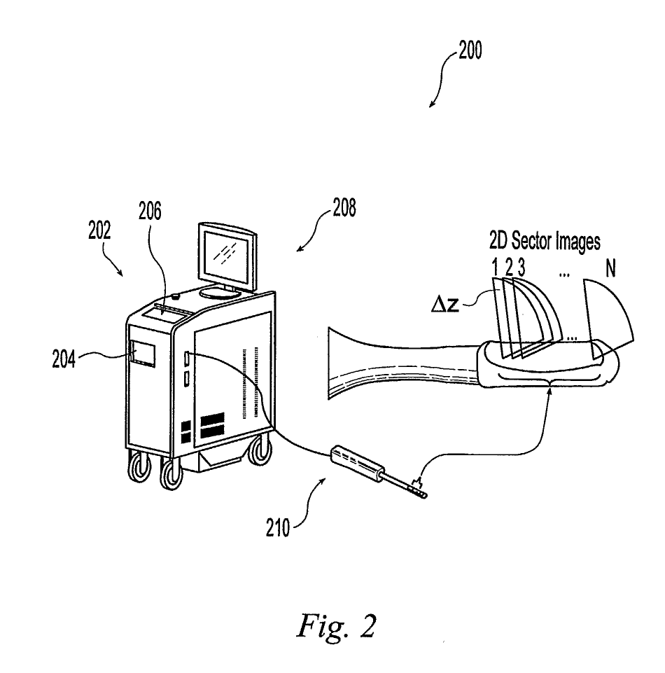

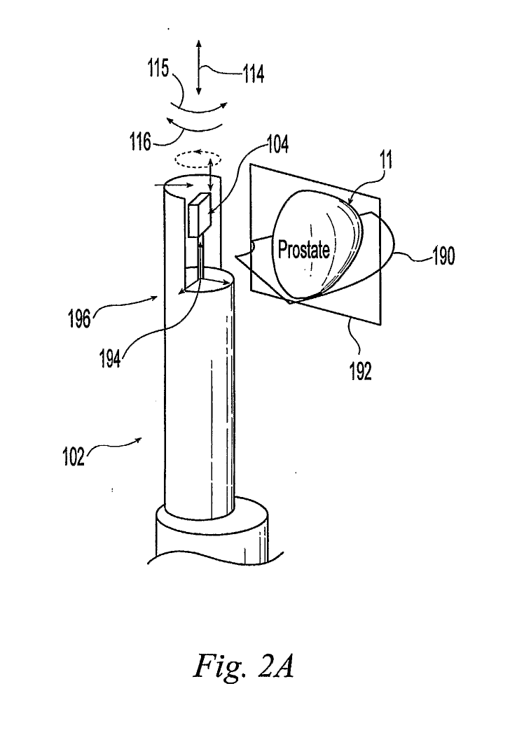

Referring to FIG. 2, an exemplary HIFU system 200 is shown, the Sonablate® 500 HIFU System available from Focus Surgery, Inc., located at 3940 Pendleton Way, Indianapolis, Ind. 46226. HIFU system 200 includes a console 202 which houses or supports a controller (not shown), such as a processor and associated software; a printer 204 which provides hard copy images of tissue 10 and/or reports; a user input device 206 such as a keyboard, trackball, and/or mouse; and a display 208 for displaying images of tissue 10 and software options to a user, such as a color display. Further shown is a probe 210 which includes a transducer member (not shown), and a positioning member (not shown). The Sonablate® 500 HIFU System further includes an articulated probe arm (not shown) which is attached to the operating room bed or surgical table (not shown). The articulated probe arm orients and supports probe 210. The Sonablate® 500 HIFU System further includes a chiller (not shown) which provides a water bath for the transducer member of probe 210 to remove heat from the transducer member during the provision of HIFU Therapy. In one embodiment, the software and/or hardware of the Sonablate® 500 HIFU System is modified to incorporate the functioning of the present invention.

Ultrasound system 100 is configured to use Doppler imaging techniques to identify the location of rapidly moving bodies in the tissue treatment area 10, such as blood flow associated with NVB 20. As explained below, such location information related to the location of blood flow is used in determining the location of various tissue components, such as NVB 20 so that these zones may be excluded from the treatment plan. In one embodiment described herein, the treatment plan is based on an automatically generated proposed treatment plan which takes into account the location of the NVB to exclude the NVB from the treatment plan.

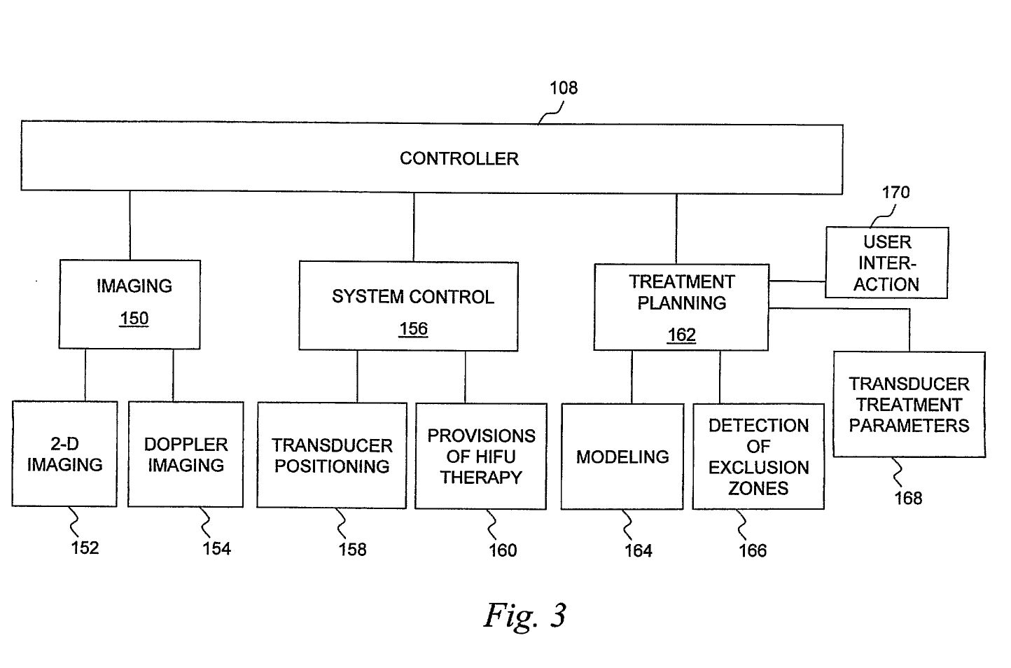

Referring to FIG. 3, an exemplary embodiment of controller 108 is illustrated. Controller 108 includes an imaging module 150 which controls the imaging of the tissue treatment area 10, a system control module 156 which controls various aspects of the system such as transducer positioning 158 and the provision of HIFU Therapy 160, and a treatment planning module 162 which develops a proposed treatment plan for treating the tissue in the tissue treatment area 10. Imaging module 150, as discussed herein, includes the imaging of the tissue treatment area by a plurality of two-dimensional images such as sector and linear images, as represented by 2-D imaging 152. Imaging module 150, as discussed herein, further includes the use of Doppler Imaging to determine the location(s) of blood flow in the tissue treatment area, as represented by Doppler Imaging 154. Treatment planning module 162, as discussed herein, generates a proposed treatment plan for treating the tissue in the tissue treatment area 10. Treatment planning module 162 includes a modeling component 164 which models tissue components in the tissue treatment area 10, a detection of exclusion zones component 166 which determines the location of exclusion zone as discussed herein, a transducer/treatment parameters component 168 which provides input on the system characteristics, and a user interaction component 170 which provides information about a proposed treatment plan to a user and receives modifications from a user.

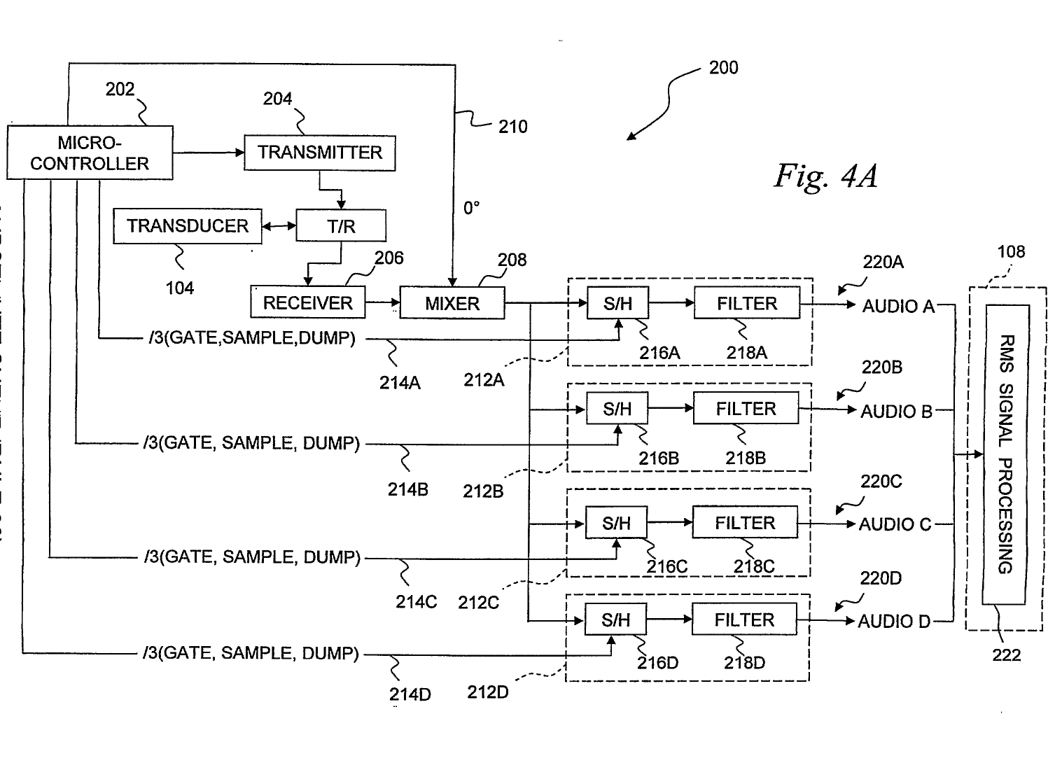

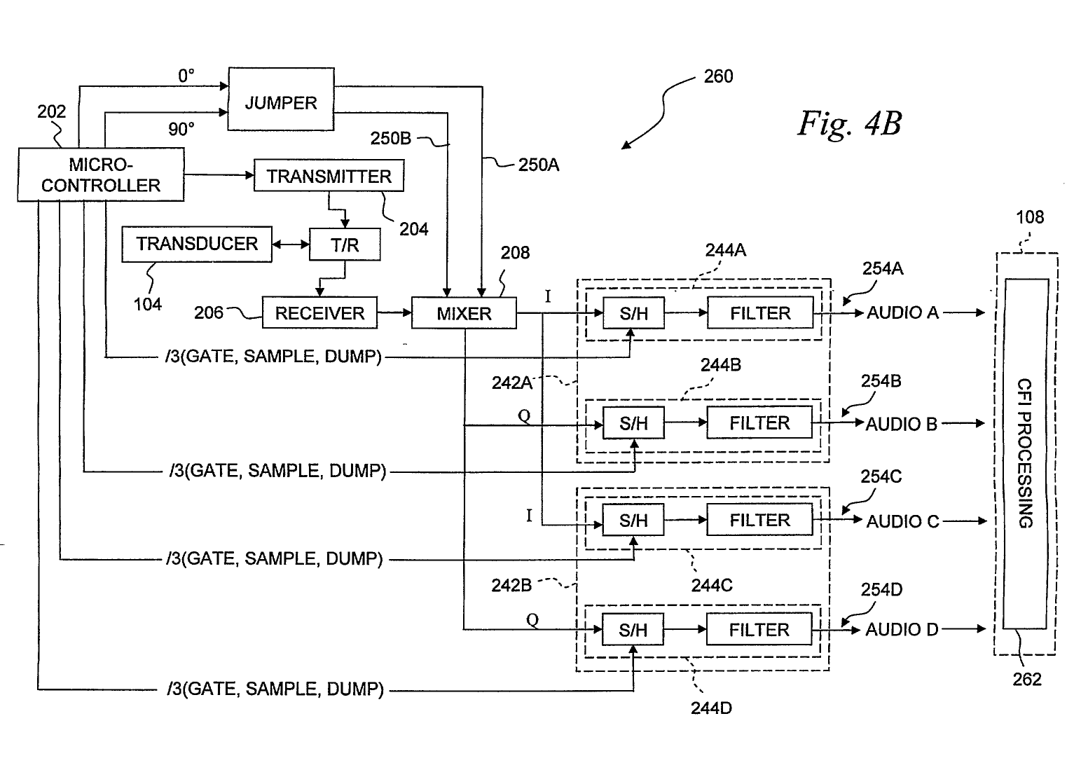

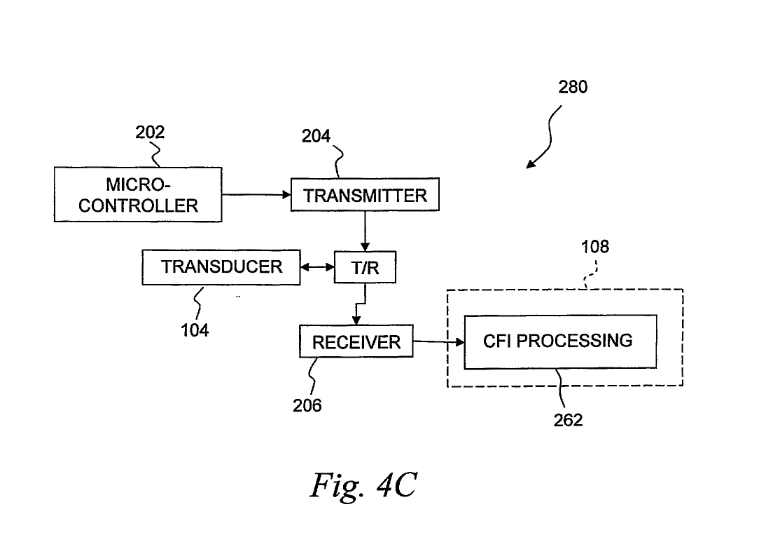

An exemplary embodiment of the Doppler System 154 of controller 108 is discussed below in connection with FIGS. 4A-C. As explained herein, the Doppler system may be implemented as software and as a combination of software and hardware. Further, a Pulsed-Wave (PW) Doppler system and a Color Flow Imaging (CFI) Doppler system are disclosed.

Referring to FIG. 4A, a first PW Doppler system 200 is shown. System 200 includes a digital micro-controller 202 which provides a signal to an analog transmitter 204 instructing the transmitter to transmit an ultrasound signal with transducer 104. In a preferred embodiment, transducer 104 uses a single element for Doppler imaging. In one embodiment, the transmitted signal is about 4 microseconds (μsec) in duration and at a frequency of about 4 MHz. Echo signals (reflected from the tissue treatment area) are received by transducer 104 subsequent to the transmitted signal and are provided to an analog receiver 206. Receiver 206 passes the received echo signals onto a mixer 208 which mixes the analog echo signal with a reference signal (indicated by line 210) from micro-controller 202. The reference signal has the same frequency as the transmitted signal and is used to remove this high frequency carrier signal from the received echo signal; thereby leaving the low frequency Doppler signal. In one example, the low frequency Doppler signal is between about 400 Hz to about 2 kHz.

The resultant Doppler signal is then provided to a series of gates 212a, 212b, 212c, 212d. The particular gate 212 that receives the resultant Doppler signal is controlled by micro-controller 202 as represented by lines 214a, 214b, 214c, 214d. In each gate (when the respective gate is active), the Doppler signal is sampled and held, as illustrated by block 216a, 216b, 216c, 216d. Further, the resultant sampled Doppler signal is filtered with a bandpass filter 218a, 218b, 218c, 218d. In one example, the bandpass filter is configured to pass frequencies in the range of about 400 Hz to about 2 kHz.

Each gate 212 is activated by microcontroller 202 for a specified period of time. For example, for an echo signal whose useful duration (the useful duration in one embodiment being the time frame generally equal to expected depth of the tissue being imaged) is n μsec, gate 212a is activated from 0 to n/4 μsec, gate 212b is activated from n/4 μsec to n/2 μsec, gate 212c is activated from n/2 μsec to 3n/4 μsec, and gate 212d is active from 3n/4 μsec to n μsec. If only a single gate 212 was used, then four separate transmitted and received echo pairs would need to be used to cover the same depth of tissue as the four gate system 200 illustrated. As such, by using multiple gates 212a, 212b, 212c, 212d more of the echo signal received by transducer 104 may be processed for the presence of Doppler information as a function of depth at the same time and hence more depth of the tissue treatment area 10 may be reviewed with a given transmitted signal and received echo pair. This reduces the amount of time needed to obtain Doppler information about the tissue treatment area 10.

In one embodiment, microcontroller 202, transmitter 204, receiver 206, mixer 208, sampling/hold 216 and filter 218 are all included on a circuit board such as an audio board. Each gate 212 contained on the circuit board having an output 220. This output 220 provides the filtered signal to one of a speaker (not shown) for auditory detection of the presence of blood flow and controller 108 for further processing. In one embodiment, controller 108 processes the output signal by root mean square (RMS) techniques, as represented by block 222, for the presence of blood flow (as indicated by the shift in frequency due to the Doppler effect). RMS processing 222 for the presence of blood flow is well known in the art and may be carried out by hardware processing and/or software processing. In an alternative embodiment, Fast Fourier Transform (FFT) spectrum integration (or power spectrum) may be used to detect the Doppler phase change. FFT typically has a higher Signal to Noise Ratio (SNR) than RMS processing.

In the above exemplary system, the microcontroller 202 manipulates the frequency and repetition rate of the Doppler transmit pulse, controls the width and depth of the multiple receiving gates 212 for analog receiving processing, and generates the reference signal 210 for demodulating the Doppler echo in analog transmit/receive circuit section. In an alternative embodiment, the multiple gates 212 of system 200 shown in FIG. 4A are replaced by software processing wherein the received echo signal is digitized and stored in memory 111 associated with controller 108. Software 109 then processes the data to separate the time signal by different gate locations. This approach requires a 16 bit or higher resolution analog to digital converter and more memory and processing capability than the above illustrated system 200.

System 200 described in connection with FIG. 4A is able to detect the presence of blood flow. However, system 200 is not able to distinguish the direction of blood flow. As illustrated in FIG. 4B, system 200 may be modified to produce system 260 which is capable of determining the direction of blood flow. System 260 unlike system 200 has two gates 242a and 242b, each having two channels 244a, 244b and 244c, 244d. However, more gates 242 may be added to system 260 such that it has an equal number of gates 242 as system 200.

System 260 has two reference signals 250a and 250b that are provided to mixer 208. Reference signals 250a, 250b are similar to reference signal 210 in that they are at the carrier frequency (in one example about 4 MHz), but reference signal 250b is 90° out of phase from reference signal 250a. Each gate 242 of system 240 samples the mixed received echo signal and 0° reference signal on a first channel (I channel) 244a and 244c, respectively, and the mixed received echo signal and 90° reference signal on a second channel (Q channel) 244b and 244d, respectively. The combination of these two channels are then processed by well known color flow imaging techniques indicated by color flow imaging routine (CFI) 262. CFI requires two channels 244 for each gate 242, both the I and Q channels. Further, multiple successive transmitted signals and their respective echo signals are required to estimate mean velocity of the flow rate of the blood. An exemplary algorithm for the estimation of the mean velocity of the flow rate of the blood is provided below:

$$Mean velocity ϖ = \frac{1}{T} {tan}^{- 1} \{ \frac{{\sum }_{i = 1}^{n} Q ( i ) I ( i - 1 ) - I ( i ) Q ( i - 1 )}{{\sum }_{i = 1}^{n} I ( i ) I ( i - 1 ) + Q ( i ) Q ( i - 1 )} \} \qquad ( 1 )$$

wherein n=number of transmitted/echo signal pairs,
T=pulse repetition interval,
I=the signal from the I channel, and
Q=the signal from the Q channel.

Referring to FIG. 4C, the multiple gate system 260 may be carried out in software, system 280, wherein the CFI Processing 262 further includes a cross-correlation function to detect the time shift of gated echoes in successive RF signal. The hardware does not require mixer 208, sample/hold circuit 216, and filters 218. All data processing is in the digital domain. However, a low signal-to-noise ration processed in the digital domain with digitization errors may affect the results of flow velocity estimation.

The location information obtained by HIFU System 100, sector and linear images and Doppler information, is used to provide on display 112a representation of the tissue treatment area 10. In one embodiment, the representation of the tissue treatment area 10 includes one or more two-dimensional views of the tissue treatment area, such as one or more sector views and one or more linear views. In one example, traditional 2-D ultrasound imaging is used to generate sectors views, such as a sector view in sector plane 190 in FIG. 2A and linear views, such as a linear view in linear plane 192 in FIG. 2A. In one embodiment as discussed herein, the displayed view, preferably a sector view, provides a representation or icon to indicate the location of blood flow as determined by using Doppler imaging techniques. In another example, multiple sector views 200 are arranged on display 112 so that the physician can see multiple sectors of the tissue treatment area 10. In still another example, both sector views and linear views are arranged on display 112 so that the physician may see multiple sectors of the tissue treatment area 10. In all of the above examples, an automatically generated proposed treatment plan, or at least a portion of the proposed treatment plan may be displayed as well.

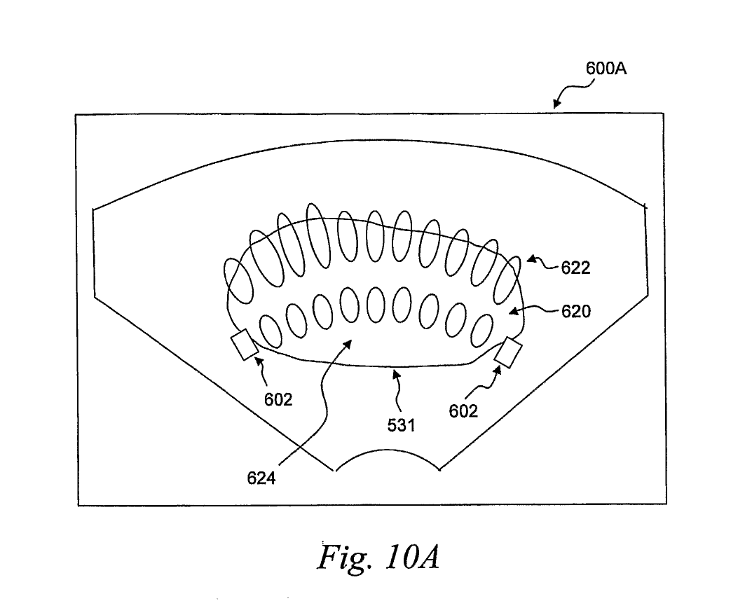

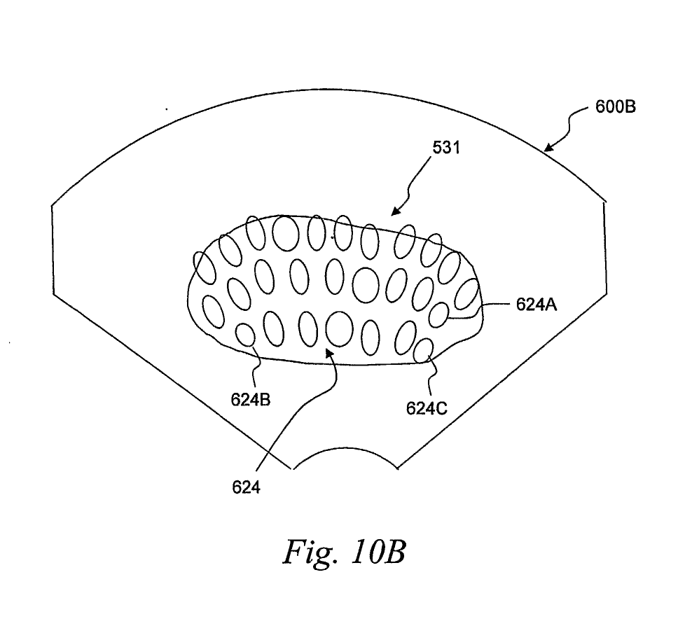

In one embodiment the icon or representation of the location of blood flow is shown as an abstracted representation, such as box 602 in FIG. 10A. This provides a general indication of the location of blood flow. The icon or representation 602 may be colored to offset itself from the background. In another embodiment, the icon or representation of the location of blood flow is one or more color pixels. The color pixels are typically colored to offset themselves from the background, such as red or blue. Further, in one example, only the pixels (either a two-dimensional pixel for a sector view or a three-dimensional pixel for a volume view) which corresponds to locations indicated as having blood flow are colored, This provides more exact location information as well as shape information of the blood flow region. In one embodiment, the brightness, color, or other indicia of the representation is an indication of the amount or velocity of blood flow or the direction of the blood flow. For instance, the representation or icon may be brighter to indicate higher volumes or velocities of blood flow.

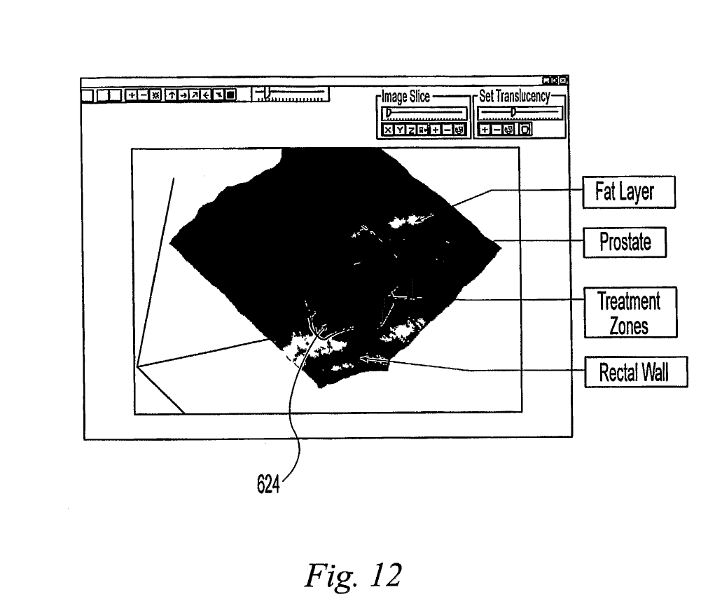

In addition to the above discussed 2-D imaging capability, in one embodiment, HIFU System 100 is configured to provide three-dimensional imaging of the tissue treatment area 10. In one embodiment, HIFU System 100 displays a volume image of the tissue treatment area 10, the volume image being generated from multiple 2-D images (see FIG. 12 for example). In a preferred embodiment, HIFU System 100 generates a three-dimensional model representation of the location of the tissue components of tissue treatment area 10. Details of a preferred method to generate three-dimensional model of tissue components in the tissue treatment area 10 is provided herein.

As described in more detail herein, various techniques are used to model the location of tissue components expected to be located in the tissue treatment area 10. In the instance wherein the tissue treatment area 10 corresponds to the area surrounding prostate 11, expected tissue components include rectal wall 18, urethra 14, prostatic capsule 12, seminal vesicles 16, and/or neuro-vascular bundles (“NVB”) 20. In one embodiment, the three-dimensional representation of one or more tissue components may be manipulated through input from user input device 110 to change the orientation of the respective three-dimensional representation. In this way a physician may virtually review all sides of the representations of the tissue components of the tissue treatment area 10.

The location information determined by the Doppler imaging, 2-D imaging and/or the 3-D imaging with HIFU System 100 in a preferred embodiment is utilized to determine an appropriate treatment procedure for treating at least a portion of the tissue in the tissue treatment area 10 with HIFU Therapy.

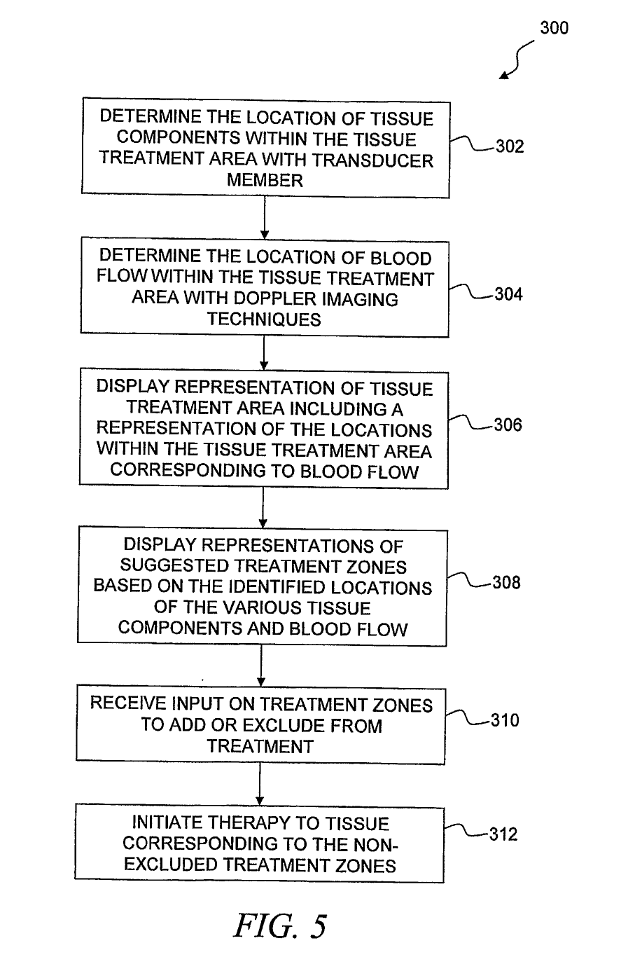

Referring to FIG. 5, an exemplary imaging and treatment method 300 is shown. In a preferred embodiment, imaging and treatment method 300 is carried by controller 108 of HIFU System 100. In one example, imaging and treatment method 300 is embodied in software 109 that is loaded onto memory 111 accessible by controller 108.

In step 302, HIFU System 100 based on ultrasound imaging data collected by transducer member 104 determines the location of tissue components located within the tissue treatment area 10. In one example, HIFU System 100 uses the 3-D modeling techniques discussed herein to determine and model the location of the tissue components, such as rectal wall 18, urethra 14, and prostatic capsule 12.

In step 304, HIFU System 100 based on Doppler imaging data collected by transducer member 104 determines the location, if any, of blood flow in the tissue treatment area 10. In one embodiment, HIFU System 100 uses the 3-D modeling techniques discussed herein to determine and model the location of the tissue components including blood flow. In another embodiment the location of tissue components is determined using both 2-D and/or 3-D imaging and Doppler imaging.

In step 306, HIFU System 100 generates a three-dimensional model of the tissue components in the tissue treatment area including the vascular components which include blood flow. The three-dimensional model is displayed on display 112.

In step 308, HIFU System 100 displays representations or indicia of proposed treatment sites based on the identified location information of the tissue components and/or the location of blood flow in tissue treatment area 10. In one example, HIFU System 100 is configured to identify various treatment zones corresponding to the location of prostatic capsule 12. In one variation, the HIFU System 100 automatically excludes locations which overlap with other tissue components from being suggested or proposed treatment zones, such as locations corresponding to NVB 20 (including blood flow). In another variation, the HIFU System 100 includes locations which overlap with other tissue components as being suggested treatment zones, such as locations corresponding to the neuro-vascular bundles 20. In this variation, the representations or treatment indicia which correspond to these overlapping locations include an indicia or icon differing from the other suggested treatment zones to alert the physician to the location of overlapping tissue (such as a differing color).

In step 310, HIFU System 100 receives input from user input device 110 related to the suggested or proposed treatment zones to add or to exclude. In some instances the physician may decide to proceed with treatment in the area of the NVB 20 to more fully treat the potential diseased tissue and/or because of the patient's wishes. It is important to highlight that the addition of Doppler imaging permits identifying the location of NVB 20, a tissue typically not resolvable by traditional 2-D imaging techniques. As such, the Doppler imaging permits the physician to have location information on the location of the NVB 20 and hence to permit the selective treatment of tissue areas based on the potential damage to the NVB 20.

In step 312, once the treatment zones have been selected and/or approved by the physician, HIFU System 100 focuses high intensity ultrasound energy at the locations in the tissue treatment area corresponding to the treatment zones. As explained more fully in U.S. Pat. No. 5,762,066 which is incorporated by reference herein, the high intensity focused ultrasound is an effective tool for selectively destroying diseased tissue surrounded by otherwise healthy tissue in a minimally invasive manner.

As is known HIFU Therapy requires the emission of a continuous wave (“CW”) for a sustained period of time with sufficient intensities to ablate the target tissue at the desired location, the focus of transducer 104. For instance, the Sonablate® 500 HIFU system typically is set to provide a CW from its associated transducer for about three seconds resulting in ablation of the target tissue at the focus of the transducer. This time period can be increased or decreased depending on the desired lesion size or the desired thermal dose.

It should be understood that the transducer member 104 of ultrasound system 100 must be capable of being repeatably positioned relative to tissue treatment area in order for the above described method to be effectively carried out. This is because registration is needed between the actual locations of the tissue components and the locations of the suggested treatment zones which are based on the location information derived from the information gathered by transducer member 104. If the transducer member 104 is not capable of being repeatably positioned than there can be no assurance that the location of a treatment zone truly corresponds to the correct location in the tissue treatment area 10.

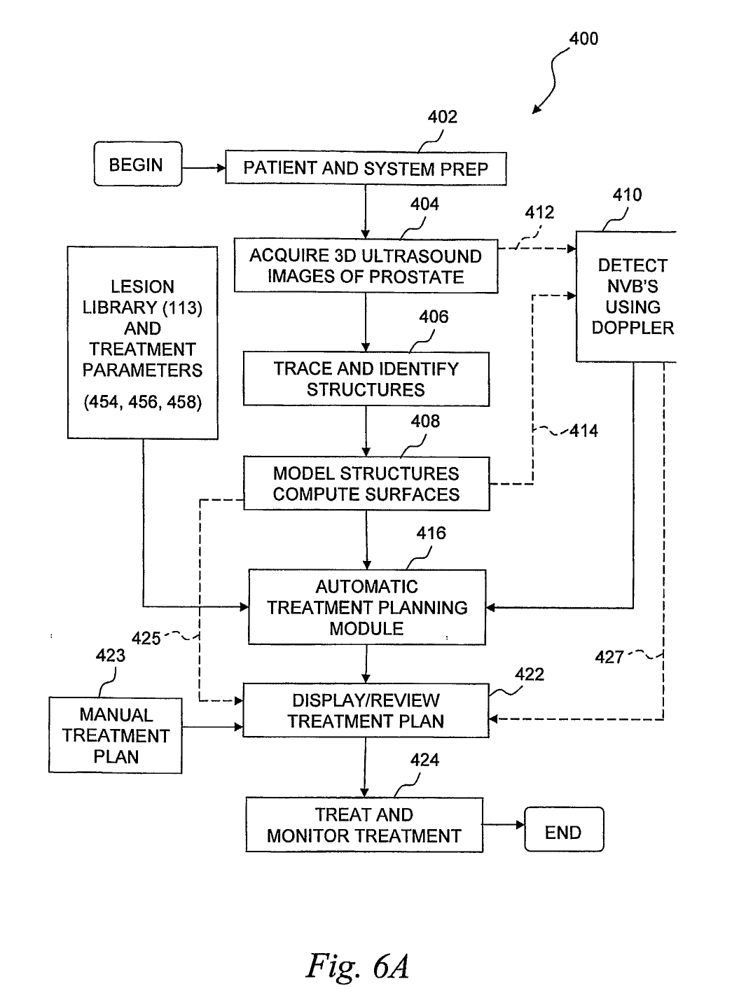

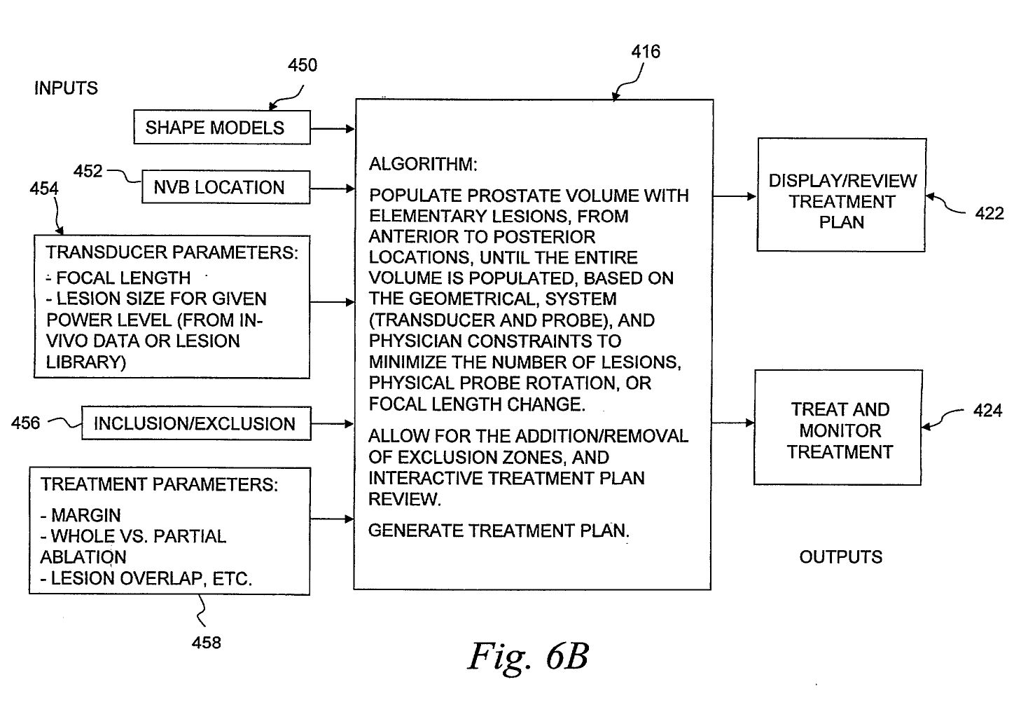

Referring to FIG. 6A, another exemplary method 400 of treating tissue treatment area 10 containing a plurality of tissue components is provided. The present exemplary method 400 is tailored to a tissue treatment area including prostate 11 and related tissue components. However, the exemplary method 400 may be used with other tissue treatment areas having other tissue components.

As represented by block 402, the patient and system 100 are setup for the treatment of tissue treatment area 10. In the case of treating prostate 11, the patient and the prostate gland are immobilized. Transducer 104 is positioned proximate to prostate 11 by the transrectal insertion of probe 102 containing transducer 104. In one embodiment, the patient is treated under general anesthesia. Probe 102 is held in place by coupling the articulated arm (not shown) to the surgical table (not shown) on which the patient is situated. In one embodiment, images of the tissue treatment area are taken with transducer 104 prior to coupling the arm to the surgical table to verify that the tissue treatment area 10 being imaged with transducer 104 includes the tissue components desired.

The tissue treatment area 10 and hence the patient should remain in the same position relative to probe 102 during the procedure. One reason for this is that the tissue treatment area 10 is imaged and these images are subsequently used to determine the portions of the tissue treatment area 10 which are to receive HIFU Therapy. Assuming that there has not been any appreciable movement between probe 102 and tissue treatment area 10, transducer 104 may be reliably positioned to provide HIFU Therapy to the correct portions of tissue treatment area 10. However, if there has been movement between probe 102 and tissue treatment area 10 then it is not possible to accurately position transducer 104 relative to tissue treatment area 10, and the patient or probe will have to be repositioned or re-aligned prior to treatment planning and HIFU Therapy.

In one embodiment, patient movement is detected by measuring the distance from transducer 104 to rectal wall 18 and to provide an indication of patient movement if that distance changes above a threshold amount. As explained herein, a plurality of sector images and linear images are taken of the tissue treatment area prior to the commencement of treatment with HIFU therapy. Further, immediately after the creation of each individual HIFU lesion (a multitude of these form the overall and complete HIFU treatment), a set of one linear and one sector image are generated (post-lesion images) and displayed with display device 112 along with the associated reference images (stored pre-treatment images) for the same site. By comparing the pre-treatment images and the post-lesion images or features of the pre-treatment images and the post-lesion images patient movement may be detected. In one embodiment, the distance from transducer 102 to rectal wall 18 in the pre-treatment images and post-lesion images are compared to provide one method of detecting patient movement.

As represented by block 404, three dimensional ultrasound images or volume images of the tissue treatment area are obtained. In one embodiment, the volume images are generated based on two-dimensional images of the tissue treatment area. In one embodiment, a plurality of sector images and a plurality of linear images are obtained. Referring to FIG. 2A, an exemplary sector image plane 190 is shown and an exemplary linear image plane 192 is shown. Also shown in FIG. 2A, is an origin 194 of a common probe space 196. The origin is defined by the center of transducer 104 when it is at its lowest position of translation in direction 114 (or the lower most aperture of a scanning aperture transducer) and when transducer 104 is pointing straight out the probe-tip. During the mathematical modeling of the various tissue components discussed herein, various intermediate data-coordinate frames are defined to simplify equations and least-squares fits, but the final equations for the modeled components are transformed back into probe space 196 for display, registration with images, and computer-generation of the treatment plan.

In one embodiment, a plurality of sector images and linear images are obtained. These sector and/or linear images are used to calculate a volume ultrasound image of the tissue treatment area. In one embodiment, the volume image is created by stacking scan-converted two-dimensional sector images. In one example, about 160 sector images, are acquired. Each sector image has 250×357 pixels. The pixel size both in the sector plane and between planes is 0.25 mm, forming cubic voxels (three-dimensional pixels) for distortion-free reconstruction and display. This forms the fundamental 3D prostate imaging dataset. This dataset spans a volume of 40 mm (long (160 pixels))×61 mm (height (250 pixels))×1100 (width (357 pixels)).

Each sector image is inserted into a 3D array in memory 111 for transfer to a rendering board which is incorporated into controller 108. The sector images are equally spaced between the proximal and distal ends of the prostatic capsule, one sector image passing approximately through the middle of the capsule; similarly, the linear images are equally spaced between the lateral limits of the capsule, one linear image passing approximately through the middle of the prostatic capsule.

Controller 108 reconstructs and displays a 3D or volume rendered view of the ultrasound data and treatment zones on display device 112. An exemplary rendering board is VolumePro™500 or VolumePro™1000 available from TeraRecon located at 2955 Campus Drive, Suite 325, San Mateo, Calif. 94403. In one embodiment, different density tissue in the volume image may be displayed in different colors with the VolumePro™1000. An exemplary volume rendering is shown in FIG. 12. Referring to FIG. 12, the prostate, rectal wall, and fat layer are visible. Further, shown in FIG. 12, are proposed treatment zones for HIFU Therapy. The formation of these proposed treatment zones is discussed herein. In one embodiment, the rendered image via input from user input device 110 may be rotated, may be scaled, and may have different image attributes, such as transparency. With the volume imaging capability, the physician or user is able to view the entire prostate 11 on the screen at one time for pre-treatment and/or post-treatment diagnosis purposes. Further, the volume imaging allows for verification of the planned treatment.

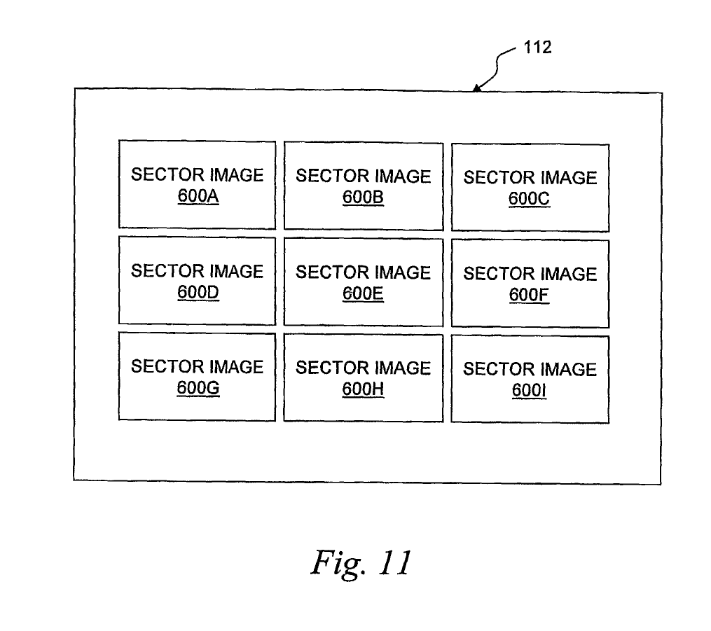

In one embodiment, the user may select to view the tissue treatment area as the volume ultrasound data shown in FIG. 12 or in multiple two-dimensional images, such as a plurality of sector images which have traditionally been used for treatment planning with the Sonablate® 500 HIFU system, such as in FIG. 11 and FIG. 11A.

In one embodiment, the volume image includes interpolated points between the various sector images and/or linear images to enhance the rendered view of the tissue treatment area and/or to reduce the number of sector and/or linear images required. In one example, bilinear interpolation, which ignores data from adjacent planes, is used. In another example, trilinear interpolation is used. In yet another example, a tri-cubic spline, is used.

The volume image data, in one embodiment, is further refined before it is presented to the physician or user for review. One exemplary refinement is adjustments to the histogram of the data to manipulate the contrast and/or brightness of the volume ultrasound data. In one example, the histogram data is set to an “S-shaped” map. Another exemplary refinement is an enhancement of boundaries in the volume data. This enhancement provides assistance to the user in tracing the prostatic capsule 12 and other tissue components in the tracing step, as represented by block 406, below. Further, this enhancement aids controller 108 in identifying the locations of various tissue components, such as rectal wall 18.

As represented by block 406, various tissue components are identified based on the volume image data of the tissue treatment area. At least some of the tissue components, such as rectal wall 18 are identified automatically by controller 108. The rectal wall boundary is automatically detected by simple edge-detection algorithms. In one embodiment, some of the tissue components are identified and located through interaction between a user and the image data, either presented as volume data or as one or more 2-D images. One exemplary tissue component located through user interaction is prostatic capsule 12.

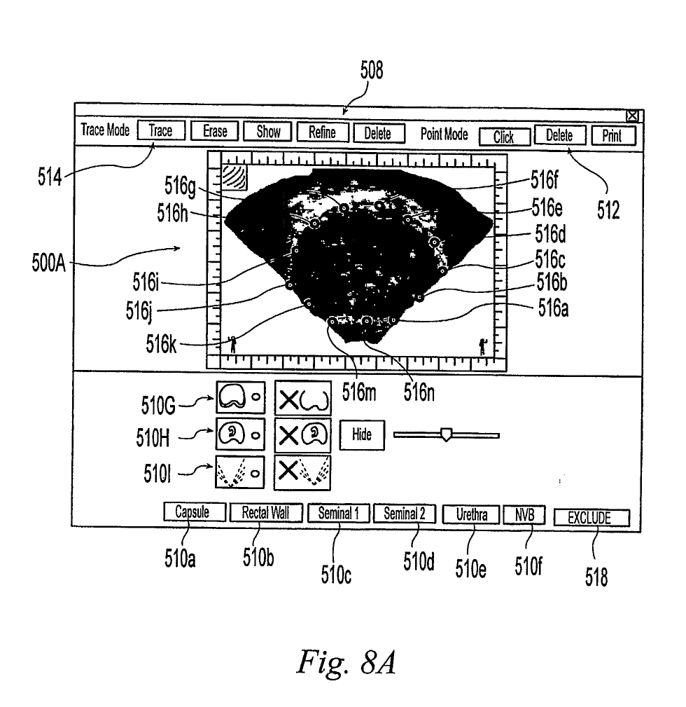

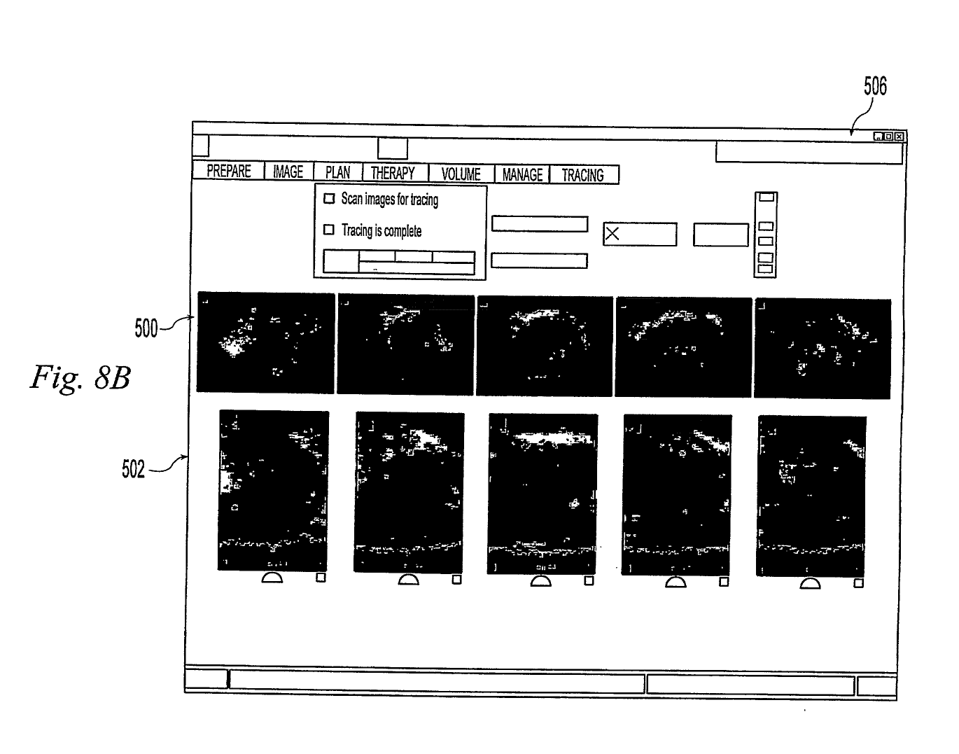

In an exemplary embodiment, illustratively shown in FIGS. 8A and 8B, the user is presented multiple sector images 500 and linear images 502 on display 112. The user then traces or otherwise marks the requested components in one or more of the sector images and/or one or more of the linear images. In the case of prostatic capsule 12, the user manually traces the contour of capsule 12 in at least one sector image (illustratively image 500A in FIG. 8A) and manually traces the contour of the capsule 12 at least one linear image. In one embodiment, the user is given up to five sector images and five linear images in which to trace the capsule (see FIG. 8B). In a preferred embodiment, the user traces at least five sector images and at least three linear images. It should be understood that additional tissue components may be traced by the user. In one embodiment, the user marks the center of urethra 14 in each sector image that the user traces capsule 12. In one embodiment, the user traces one or more of urethra 14, seminal vesicles 16, and rectal wall 18, in addition to the prostatic capsule 12.

In one embodiment, for each anatomical structure, the user defines the tracing of a boundary by clicking on points to define a rubber-banding B-spline fit on slices in any or all of the multiple orthographic views. Typically, in the case of prostatic capsule 12 the user will trace the views near mid-gland and at equally spaced intervals to encourage more uniformly spaced data for unbiased geometric model fits.

In addition to tracing the boundary of capsule 12 in each sector image 500, the user is to mark the center of urethra 14 in each sector image 500. In one embodiment, a sonolucent catheter is inserted into urethra 14 during the imaging process to make urethra 16 easier to identify in sector images 500. The catheter is removed prior to HIFU Therapy being administered to the tissue treatment area. The trace data for capsule 12 and urethra 16 are recorded as xyz-Cartesian coordinate data in the probe reference space 196 and written to memory 111 for use in modeling.

An exemplary user interface for tracing or otherwise marking tissue components is shown in FIGS. 8A and 8B. The interface 506 of FIG. 8B provides a plurality of sector views 500 and linear views 502 which the user may select to trace boundaries thereto. The interface 508 of FIG. 8A is a trace mode screen wherein the user is presented with an image, illustratively image 500A, on which the user traces or marks appropriate portions of the images 500A. The user is able to select a tissue component to be marked from the plurality of tissue components listed at the bottom of the screen by selecting the corresponding textual button (capsule 510A, rectal wall 510B, seminal vesicles 510C, 510D, urethra 510C, and NVB 510F) or by selecting the corresponding iconic button (capsule 510G, urethra 510H, and seminal vesicles 510I). Further, as shown in FIG. 8A, the user is able to mark components in a point mode 512 and a trace mode 514. In the point mode, the user places points to define the outline of the component (illustratively points 516a-n to define the outline of capsule 12) or the center of the component (in the case of the urethra). Controller 108 then uses the points to generate traces, such as b-spline traces. In the trace mode, the user outlines the region by tracing a closed line (i.e. by holding down a mouse button during the trace). The controller 108 then takes this information to generate a smoother trace.

Further, as explained in more detail below, the user may desire to exclude certain regions of the tissue from treatment. Some of these regions are automatically identified by the system, such as NVB 20 as explained herein. Other regions are identified by the user. One method of identifying these region is to trace or otherwise mark these regions similar to the tracing of tissue components. For instance, the user may select an exclude button 518 and provide trace data for regions that the system should exclude from treatment. One example, may be ejaculatory ducts which are typically not detected by ultrasound, but their location may be inferred by the user from other structures.

The trace data, other marking data (urethra centers), and automatically located boundaries (such as the rectal wall 18) are used to develop three dimensional models of at least some of the tissue components in the tissue treatment area 10, as represented by block 408. Referring to FIG. 6, the following exemplary models are shown: prostatic capsule 530, urethra 532, rectal wall 534, and seminal vesicle 536. These three-dimensional models are used by system 100 in the automatic generation of a proposed treatment plan, as discussed herein, to assist in the location of other tissue components (such as NVB), to remove clutter from the volume ultrasound data, and/or to provide a visual representation of tissue treatment area 10. Many different techniques may be used to model the tissue components in the tissue treatment area. Exemplary methods of modeling the prostatic capsule 12, the urethra 14, and the rectal wall 18 are provided in the attached APPENDIX and/or in U.S. Provisional Application Ser. No. 60/568,556, filed May 6, 2004, which is expressly incorporated by reference herein.

In one embodiment, urethra model 532 is generated by modeling urethra 14 as a parametric tube of the form:
xᵤᵣₑₜₕᵣₐ(t,θ)=h(t)+R cos θ (2a)
yᵤᵣₑₜₕᵣₐ(t,θ)=h(t)+R sin θ (2b)
zᵤᵣₑₜₕᵣₐ(t,θ)=t (2c)
This circular cylinder is modeled with a constant radius R, such as R=2.5 mm. This radius size should envelope almost all real urethras. The path h(t) is found by performing a least squares fit to the centers of the urethra identified or marked by the user in the various sector images 500.

In one embodiment, rectal wall model 534 is generated by modeling rectal wall 18 of the form:
x_{rectalwall}(t,θ)=R(t)cos θ (3a)
y_{rectalwall}(t,θ)=R(t)sin θ (3b)
z_{rectalwall}(t,θ)=t (3c)
Rectal wall 18 is assumed to have a linear axis which corresponds to the probe axis (as indicated in FIG. 2A by direction 114) and a variable radius R(t). The radius function R(t) is determined by performing a least squares fit to the radii of the circles that best fit rectal wall 18 in each sector image 500.

In one embodiment, the left- and right-seminal vesicles are modeled as unions of overlapping spheres of varying radii fit to user-defined boundaries. In order to mark these components the user traces their boundaries, similar to the tracing for the prostatic capsule.

Prostatic capsule 12 may be modeled by various techniques. In one embodiment, prostatic capsule may be modeled as approximating a sphere. In a further embodiment, prostatic capsule 12 may be modeled as an ellipsoid. In another embodiment, prostatic capsule model 530 is generated by modeling prostatic capsule 12 with Fourier ellipsoids. A Fourier ellipsoid is obtained by replacing the (elliptical) cross-sections normal to the major axis of a standard ellipsoid by curves that have a more general Fourier description. This permits the resultant surfaces to have more complex spatially-varying geometric features than standard ellipsoids. This is particularly advantageous when dealing with diseased and/or clipped prostatic capsules whose shape may be irregular.

In one embodiment, the parametric equations for the Fourier ellipsoid are of the form:
x_{capsule}(t,θ)=F(t,θ)cos θ (4a)
y_{capsule}(t,θ)=F(t,θ)sin θ (4b)
z_{capsule}(t,θ)=t, (4c)
where t ε [−1, 1], θ ε [0, 2π], and F(t, θ) is a truncated Fourier series:

$$F ( t , θ ) = {( 1 - {t}^{2} )}^{\frac{1}{2}} 〈 {f}_{o} ( t ) + {\sum }_{n = 1}^{N} ( {f}_{n} ( t ) cos ( n θ ) + {g}_{n} ( t ) sin ( n θ ) ) 〉 , \qquad ( 5 )$$

with polynomial blending f₀(t), and fₙ(t) and gₙ(t), along the linear axis of the capsule. Based on equations 4a-c, the shape of prostatic capsule 12 may be determined along with other parameters such as the volume of prostatic capsule 12, the surface area of prostatic capsule 12, and the relative location of points within the treatment area as being either inside or outside of prostatic capsule 12 or on the surface of prostatic capsule 12. In one embodiment, the surfaces for the prostatic capsule model 530 are used to generate a solid volume model of the prostate. Additional details concerning the modeling of the prostatic capsule are provided in the APPENDIX.

As represented by block 410, Doppler imaging is used to determine and/or assist in determining the location of various tissue components within the tissue treatment area 10, In an embodiment, wherein the prostate is to be treated, Doppler imaging is used to locate NVB 20 so that these nerves may be excluded from treatment. NVB 20 resides close to the surface of prostatic capsule 12 and are densely vascularized. Treatment of NVB 20 with HIFU Therapy may result in impotency, erectile dysfunction, and/or incontinence. However, the user may still decide to treat these regions if the user feels that cancer is in close proximity to NVB 20.

In one embodiment, Doppler imaging data is generated with transducer 104 separate from the generation of the two-dimensional sector and linear images (discussed in relation to block 404). In one example, the two-dimensional sector and linear images are obtained prior to the Doppler information. In one embodiment, as illustrated by dashed line 414 the system uses the location of the shape models to determine portion of the tissue treatment area 10 to scan during the Doppler imaging. This is because NVB 20 are typically in a given spatial relationship to other tissue components such as prostatic capsule 12. Also, in one embodiment, as illustrated by dashed line 412, either the two-dimensional or the volume ultrasound data may provide the location of various tissue components and hence be used to determine the portions of the tissue treatment area to scan during Doppler imaging. As stated herein it is well known in the art to use Doppler imaging techniques to determine the location of blood flow.

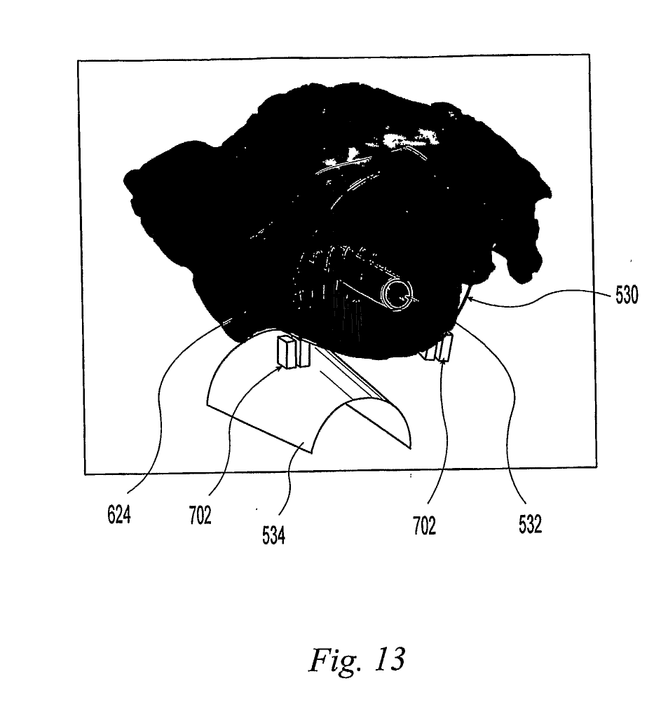

Portions of tissue treatment area 10 exhibiting blood flow may be displayed with display device 112 with a special representation or icon. As shown in FIG. 11A, the blood flow may be shown on two-dimensional sector images 600A as icons 602. Further, the blood flow may be shown as an overlay on top of the volume ultrasound image and/or tissue component models via a color-map (see FIG. 13, blood flow icons 702). The color map, in one embodiment color codes the displayed representation or icon to provide an indication of the amount of blood flow present. The display of the blood flow enables the physician or user to visualize the position of the blood flow and its relative position to other tissue components, such as the prostatic capsule (prostatic capsule model 530 in FIG. 13). Further, the physician or user, based on the amount of blood flow present, may be provided an indication of the health of NVB and whether NVB 20 is healthily enough to warrant exclusion from HIFU treatment.

In one embodiment, Doppler imaging is used to detect very small prostate cancer sites. In the early stages of cancer growth the cells form neo-vascularization. Therefore, Doppler imaging may be used to determine the location of these sites. Unlike the case of NVB, these sites are targeted for treatment with HIFU Therapy. In one embodiment, an ultrasound contrast agent is used to enhance the detection of these sites and/or NVB 20.

As explained herein, the Doppler imaging not only provides a visual cue to the user, but it also is one of a plurality of input to an automatic treatment planning module 416 which develops a proposed HIFU treatment plan for review by a user. Traditionally, the Sonablate® 500 HIFU system requires a physician or user to define treatment zones on up to 15 different ultrasound images that span the entire prostate. In one embodiment, HIFU System 100 is configured to generate a proposed HIFU treatment plan consisting of a plurality of treatment zones without the need of input from the user except for desired modifications to the proposed HIFU treatment plan made by the user and the marking of tissue components as discussed herein in connection with block 408.

As represented by block 416, an automatic proposed treatment plan is developed to treat portions of tissue treatment area 10. Referring to FIG. 6, the automatic treatment module 416 uses the following five categories of inputs in developing the automatic proposed treatment plan: shape models 450 generated during step 408, location information about the NVB 452 generated during step 410, transducer parameters 454, Inclusion/Exclusion information 456, and treatment parameters 458. Shape models and NVB location information has been discussed above.

Transducer parameters 454 play an important role in the development of an automatic proposed treatment plan. Exemplary transducer parameters 454 include focal length of the transducer, the size of the transducer, and the degree of rotation by the transducer (in one example the transducer may be rotated 110° and still transmit and receive ultrasound energy through a window in the probe housing).

The size and shape of a single thermal lesion produced as a result of HIFU Therapy to a given treatment site is governed by the geometry of the transducer 104 within transrectal probe 102, the duty cycle and repetition rate of the applied acoustic signal, the acoustic properties of intervening tissue types, and the acoustic power delivered at the focus. As explained herein, the present application is not limited to a particular type of transducer 104. However, for illustrative purposes it is assumed that transducer 104 is a spherically focused, truncated spherical shell transducer with a 30 mm diameter aperture and having two transducer faces, one face having a 30 mm focal length operating at 4 MHz and about 30 W of total acoustic power and the other face having a 40 mm focal length operating at 4 MHz and about 37 W of total acoustic power. This is similar to the transducers traditionally used with the Sonablate® 500 HIFU System. Ultrasound exposures for a given HIFU Therapy are assumed to be about 3 sec. HIFU “ON” followed by about 6 sec. HIFU “OFF” duty cycle.

At the transducer and acoustic signal parameters provided (3 Sec ON, 4 MHz, 30 W TAP) the dimensions of a single thermal lesion are generally ellipsoidal, approximately 3 mm in width, and approximately 10 mm in length. Further, the thermal lesion is located near the geometric focus of transducer 104. In addition, these elliptical thermal lesions when spaced about 2-3 mm apart tend to merge via thermal diffusion to form a larger necrotic volume. In one embodiment, about 1000 thermal lesions are needed to treat an average human prostate.

The acoustic properties of the intervening tissues are patient specific and temperature dependent. However, the variation in these properties do not substantially change the initial deposition of an isolated thermal lesion from that predicted by an elementary lesion. Thus, the size and shape of a thermal lesion is mainly controlled by the electrical power applied to transducer 104 and the geometry of the transducer, and its subsequent conversion of electrical power to acoustical power.

By varying the focal length of transducer 104, such as with a phased-array transducer, and/or varying the electrical power applied to transducer 104 the size and location of a resultant thermal lesion may be controlled. As such, in areas of the tissue treatment area 10 proximate to critical tissue components, such as the rectal wall 18 or urethra 14, a larger number of low power thermal lesions may be planned compared to other portions of the tissue treatment area 10 such as in the main portion of the prostatic capsule 12. Therefore, detailed treatment plans having pre-defined control of the HIFU dosage (i.e. intensity times exposure time) make it possible to shape thermal lesion patterns (like sculpturing) to increase efficacy and reduce side effects, especially when treating close to rectal wall 18. Also, as explained below these detailed proposed treatment plans may be initially automatically developed and provided to physicians or users for review without requiring the physician to designate treatment sites or zones.

In one embodiment, a lesion library 113 is created wherein lesion size is categorized based on one or more parameters, such as focal length of transducer 104, excitation energy of transducer 104, HIFU on-time of transducer 104. As such, if the automatic treatment plan module 416 requires a small size lesion, one or more ways of generating such a lesion may be determined based on lesion library 113.

In one embodiment, lesion library 113 is based on in-vivo observations of the size of lesions produced with known parameters, is based on simulated lesions, and/or a combination thereof. In one embodiment, simulation software based on solving the transient bio-heat transfer equation (BHTE), as is well known in the art, is used to simulate various thermal lesions and hence to populate the respective lesion library 113.

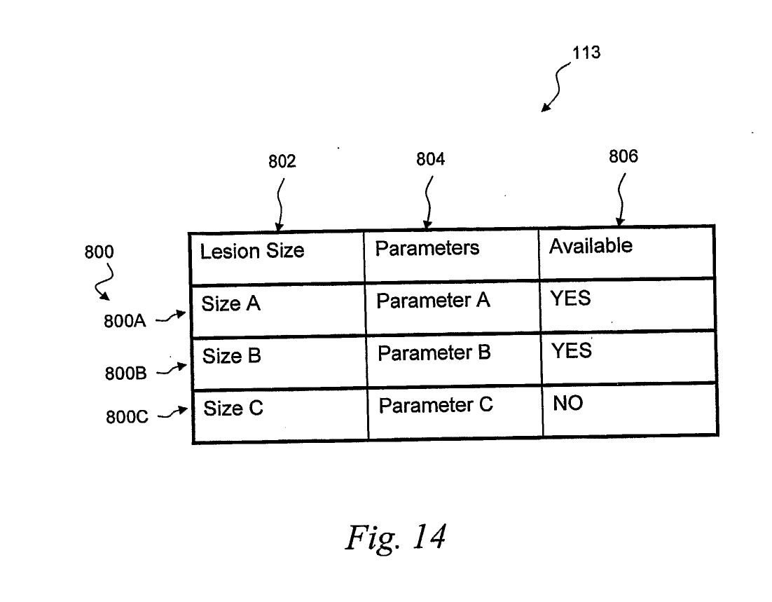

Referring to FIG. 14, a representation of one embodiment of lesion library 113 is shown. Lesion library 113 includes a plurality of exemplary lesions 800, illustratively three 800A-C, which may be used by the automatic treatment module 416 to develop a proposed treatment plan as explained herein. For each lesion 800, a lesion size 802 is provided. Lesion size 802 provides an indication of the expected size of a lesion produced during HIFU Therapy and is based on either in-vivo observations or simulations. In addition, associated parameters 804 are provided for each lesion 800. Parameters 804 are the parameters required to produce the respective lesion 800 and may include transducer, focal length, center frequency of CW, HIFU ON-time, HIFU OFF-time, transducer aperture, power levels, and water standoff distance (the distance between the transducer face and the rectal wall. This distance influences the ultimate size of a lesion as the ultrasound wave is not appreciably attenuated while traveling through water. Thus, for a large water standoff (required to treat close to the rectal wall), larger lesions are generated compared to a small water standoff (required to treat deep in the prostate) using the same power settings). In addition, a status 806 is provided. Status 806 provides an indication whether the respective lesion is available for selection by the automatic treatment module 416. Examples wherein a particular lesion, illustratively lesion 800C, would not be available include situations wherein the transducer used to generate lesion 800C is not currently either coupled to the HIFU System or is otherwise unavailable. For example, lesion 800C may be generated with a 35 mm focal length transducer and only a 30 mm focal length transducer and a 40 mm focal length transducer are available.

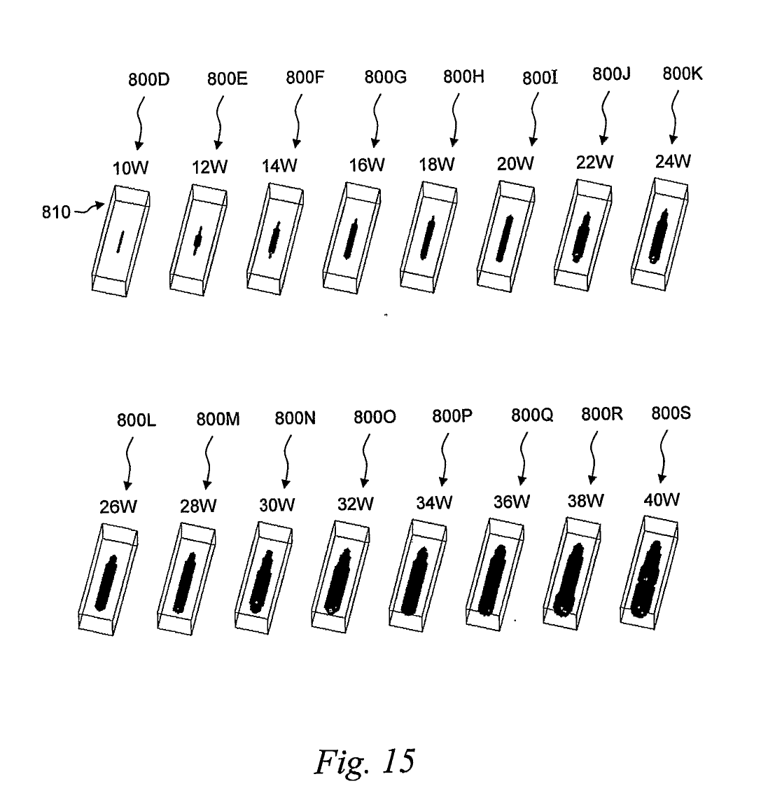

Referring to FIG. 15, representative lesions 800D-S from lesion library 113 are shown. Each of lesions 800D-S are generated with a 40 mm focal length transducer with a 15 mm water standoff. Further, the differences between lesions 800D-S are generated by varying the power level provided at the proposed treatment site. The power level for each lesion 800D-S is provided in FIG. 15 along with a reference box 810 which is the same for each lesion 800D-S and has dimensions of 4×4×12 mm. As shown in FIG. 15, a wide variety of lesion sizes may be generated. As such, lesions 800D-S provide a wide variety of sizes that automatic treatment planning module 416 may select to develop the treatment plan for the tissue treatment region 10.

In addition, various treatment parameters 458 may be defined as an input to the automatic treatment module 416. These treatment parameters are provided by the physician. In one embodiment, the physician is prompted to enter these parameters. One exemplary treatment parameter is margin. Margin is a percentage of a thermal lesion that may cross the boundary of the prostatic capsule into neighboring tissue. Another exemplary treatment parameter is Whole vs. Partial Ablation which relates to whether or not it is desired to treat the entire capsule or only a pre-determined zone, for example. In one embodiment, the predetermined zone may be traced by the physician similar to the capsule. Yet another treatment parameter is Lesion Overlap which relates to the percentage, if at all, the physician desires adjacent treatment sites to overlap each other. Other treatment parameters will be known to those skilled in the art.

These inputs are used to generate an automatic treatment plan for the treatment of diseased tissue in the tissue treatment area 10, such as prostatic capsule 12 in the case of BHP and prostate cancer. The automatically generated treatment plan significantly reduces the overall time required to develop a treatment plan to treat the diseased tissue in the tissue treatment area 10 and provides a valuable decision aid for the physician or user near critical structures, such as rectal wall 18 or NVB 20.

Based on the discussed inputs an automatic treatment plan is generated with the automatic treatment planning module 416. The following discussion assumes that prostatic capsule 12 is to be treated for prostate cancer. However, as stated herein the techniques discussed herein may be used to treat various types of diseased tissues in various tissue treatment areas 10, whether the tissue treatment area includes prostate 11 or not. As such, the disclosed systems 100 and methods should not be limited to only the treatment of prostate 11 and diseases of the prostate.

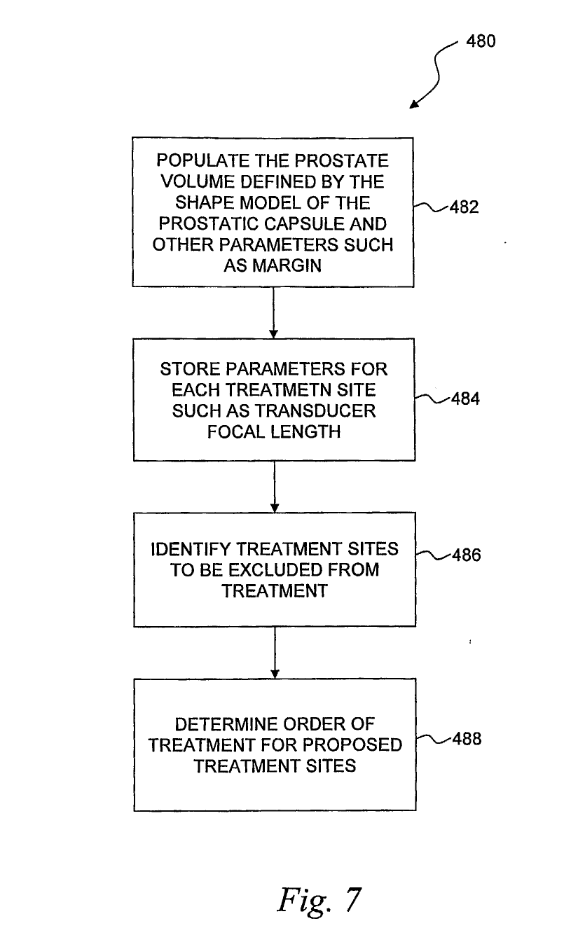

Referring to FIG. 7, an exemplary algorithm 480 for automatic treatment planning module 416 is illustrated. As represented by block 482, proposed treatment sites (proposed locations for thermal lesions) of a given size are positioned such that the entire prostatic capsule model 530 (and slightly beyond the prostatic capsule model 530 based on a value of the margin parameter) is subject to treatment with HIFU Therapy. In a preferred embodiment, the placement of the proposed treatment sites are restricted to portions of the tissue treatment area which were previously imaged during the acquisition of the two-dimensional images as discussed in connection with step 404. In one embodiment, the center of each lesion is proposed to be located at a point in the tissue treatment area 10 which is imaged in at least one sector image and at least one linear image.

FIGS. 10A and 10B show exemplary indicia of proposed treatment sites 624 indicating the exemplary proposed treatment sites for a respective given sector image 600a and 600b. The size and shape of these exemplary indicia of the proposed treatment sites 624 are defined in the lesion library 113. The position of each indicia of proposed treatment sites 624 and hence the position of the resultant actual lesion is determined by the automatic treatment planning module 416. FIG. 10A illustrates generally two radial rows 620 and 622 of treatment sites (for a transducer having two focal lengths) and generally constant size thermal lesions or treatment sites within a given row of treatment sites 620, 622. FIG. 10B illustrates treatment sites 624 at at least three focal lengths and having varying sized lesions. In particular smaller lesions are used to fill in portions near the edge of the prostatic capsule model 530, such as lesion 624a, and proximate to rectal wall 18, such as lesions 624b and 624c. It should be understood that the shown treatment sites do not illustrate the cumulative effect of heating the surrounding tissue. In a preferred embodiment, the spacing of the proposed lesions is chosen such that the individual lesions merge to form a larger lesion.

In one embodiment, larger lesion sizes are proposed where appropriate such as away from the rectal wall 18. By using larger lesion sizes the overall number of lesions may be reduced and hence the overall procedure time is reduced.

In one embodiment, the automatic treatment module 416 develops the proposed treatment sites as follows. First, the parameters provided by the physician are used to determine the area of the prostatic capsule and potentially areas proximate to the prostatic capsule to treat, such as the Margin parameter, and the desired spacing of the lesions, such as the Lesion Overlap parameter.

Once the area of the capsule has been determined, the automatic treatment module 416 selects proposed lesions 800 from lesion library 113. The process is similar to filling ajar with rocks or blocks. First, a gross filling of the jar is completed by placing large rocks or blocks in the jar. Subsequently, a fine filling of the jar is completed by placing smaller rocks or blocks, even sand, in the jar. The smaller rocks or blocks fill in the spaces left open by the large rocks or blocks. In a similar way, the automatic treatment module 416 performs a gross filling of the area to be treated by filling the region with large size lesions 800 from lesion library 113. Next, the automatic treatment module 416 performs a fine filling of the area to be treated by filling the open regions of the area with smaller lesions 800 from the lesion library 113 In contrast to the jar illustration, the automatic treatment module 416 also limits the portions of the area which are filled in the gross filling based on the tissue components in that region, such as near the rectal wall.

As may be appreciated, lesion library 113 provides a plurality of different size blocks or “brushes” for the automatic treatment module 416 to use in planning a proposed HIFU treatment. As explained herein, the size of the blocks or brushes is dependent upon the system parameters including the transducer parameters. In one embodiment, lesion library includes two brushes or blocks, one for a 40 mm transducer at a total acoustic power of 37 watts (W) and one for a 30 mm transducer at a total acoustic power of 20 W. In another embodiment, lesion library has approximately 60 brushes which are generated by varying the total acoustic power (see FIG. 15 for example) for either the 40 mm transducer or the 30 mm transducer or both. In addition, by adding additional transducers to the HIFU system or using a phased array capable of focusing HIFU energy at various focal depths the number of blocks or brushes may be increased.

In addition, to placing the proposed tissue treatment areas system 100 stores the required transducer parameters 454 and/or treatment parameters 458 for each proposed treatment site as represented by block 484. In one embodiment, these parameters are stored in the lesion library 113 along with the size of each lesion 800. Therefore, system 100 determines the required focal length of the transducer 104, the acoustic signal properties, and so forth. These parameters are used during the actual treatment of the tissue treatment area with HIFU Therapy. The automatic treatment module 416 also notifies the user of any additional setup requirements, such as repositioning probe 102 to treat various portions of prostate 11.

As represented by block 486, the automatic treatment plan also automatically removes or deactivates proposed treatment sites based on their proximity to various tissue components such as rectal wall 18 and/or NVB 20. In one embodiment, these treatment sites are not even originally proposed to the physician. In another embodiment, these treatment sites are originally proposed and subsequently deactivated. In one example, the physician selects with user input device 110 the treatment indicia 624 corresponding to the treatment sites the physician desires to remove or deactivate. Further, the physician may select with user input device 110 treatment sites to add to the proposed treatment plan.

As represented by block 488, the automatic treatment plan determines the order of treatment for the proposed treatment sites. In one embodiment, this proposed order of treatment is determined prior to the proposed treatment plan being submitted to a physician or user for review. In another embodiment, the order of treatment for the proposed treatment sites is determined after the physician or user has approved the proposed treatment plan, but prior to commencement of HIFU Therapy to any of the treatment sites. In one exemplary treatment plan, treatment sites in a given sector plane are treated prior to the treatment of treatment sites in another sector plane. Further, treatment sites further from the transducer are treated prior to treatment sites proximal to the transducer (in the case of the prostate anterior to posterior treatment) due to the fact that HIFU therapy changes the properties of tissue in such a way that it prevents tissue ablation behind already ablated tissue.

Returning to FIG. 6, prior to treating the tissue region with HIFU Therapy, the proposed treatment plan is presented to the physician or user for review, as represented by block 422. One example of the presentation of the proposed treatment plane is shown in FIG. 11. In FIG. 11 a plurality of sector images 600a-i are shown with display device 112, each sector image 600 containing a portion of the proposed treatment sites, are displayed with the display device 112. A representation of one of the sector images is shown in FIG. 10A wherein an outline 531 of the shape model 530 for the prostatic capsule is shown, along with the proposed treatment sites shown as a plurality of treatment indicia 624 in rows 620, 622. Further shown are icons or blood flow representations or indicia 602 representing regions of blood flow detected with the Doppler imaging, these regions being associated with NVB 20. As stated herein, in one embodiment, the appearance of representation or indicia 602 indicating the regions of blood flow also include a color indication of the amount of blood flow so that the physician or user may make a determination as to the health of NVB 20. Ultrasound imaging data (not shown) for the given sector is also shown in FIG. 10A. By displaying multiple sector images on the screen at the same time with the automatically generated proposed treatment plan, the physician is able to easily review the proposed treatment plan.

The display shown in FIG. 11 in one embodiment includes buttons, slider controls, or other inputs which permit the selection of the number of sector images to be shown with display. Exemplary arrangements of sector images include 2×2, 3×3 (as shown), and 4×5 matrix display formats. Further controls may be included to select sector slice spacing (0.5, 1, 2, 3, and 3.8 mm), select size and define treatment zones at two different treatment depths, step through a sub-set of sector slices in higher resolution, and measurement functions (to provide measurements such as from a treatment site to a tissue component such as the urethra). Indicators showing the treatment zones, transducer focal limits, and rectal wall location may be enabled as graphic overlays on each slice (similar to the overlay for the prostatic capsule shown in FIG. 10A).

The physician may also select to view the indicia of the proposed treatment sites 624 as an overlay to the volume ultrasound data. An exemplary representation of such a display is shown in FIG. 12.

The physician also may select to view the proposed treatment sites 624 as an overlay to one or more of the shape models (such as prostatic capsule 530) and/or the volume ultrasound data. The surfaces of the shape models are rendered as thin, almost transparent, shells so the prostate anatomy (volume ultrasound data) and volume treatment zones can be clearly visualized for verification of the treatment plan. In one embodiment, the user is able to interactively select which shape models are to be viewed. In another embodiment, the system 100, limits the amount of volume ultrasound data to be displayed to portions of the tissue treatment area which are inside the prostatic capsule shape model and generally proximate to the outer surface of the prostatic capsule. An exemplary representation of such a display is shown in FIG. 13. In addition, the physician may rotate images on display 112, zoom images on display 112, and slice the images on display 112 through a touch screen input device.

As discussed herein, the physician is further able with user input device 110 to select further regions of the treatment zone for inclusion in the proposed treatment or for exclusion from the proposed treatment. In one embodiment, all of the physician's selections discussed herein are made through a touch screen interface of display 112 and additional input devices 110, such as a keyboard, as needed. Once the physician is satisfied with the proposed treatment plan, the proposed treatment plan is approved, by input from the user (such as selecting a button to approve treatment). Finally, the proposed treatment plan is executed by the system once the order of treatment of the treatment sites has been determined, as represented by block 424.

In one embodiment, automatic treatment planning module 416 is not used or available. In this embodiment, the physician develops a manual treatment plan, as represented by block 423. The manual treatment plan may be laid out by the physician on a plurality of two-dimensional images, such as sector images and/or linear images. The display and review function 422 may still be used to display the manually generated treatment plan and to review the treatment plan. All of the same viewing options are still available with the display of the manual treatment plan, as indicated by line 425 for the shape models and line 427 for the blood flow information. As such, the physician may use the blood flow information and the shape models in the review of the manual treatment plan to determine if modifications are required.

Although the invention has been described in detail with reference to certain preferred embodiments, variations and modifications exist within the spirit and scope of the invention as described and defined in the following claims.

1. A method of providing treatment to a tissue treatment area including a plurality of tissue components, the method comprising the steps of:
generating ultrasound data related to the tissue treatment area; and
automatically generating a proposed treatment plan of the tissue treatment area, the proposed treatment plan including a plurality of treatment sites selected to receive HIFU Therapy, the plurality of treatment sites being selected based on a three-dimensional model of a first tissue component located in the tissue treatment area, the three-dimensional model of the first tissue component being based on the generated ultrasound data.

2. The method of claim 1, further comprising the steps of:
detecting blood flow in the tissue treatment area; and
excluding a first portion of tissue from the proposed treatment plan, the exclusion of the first portion of tissue being based on the detection of blood flow at a location generally corresponding to the first portion of tissue.

3. The method of claim 2, wherein the exclusion of the first portion of tissue is further based on the location of the first portion relative to the three-dimensional model of the first tissue component.

4. The method of claim 2, wherein the tissue treatment area generally corresponds to a prostate of a patient, the first tissue component corresponds to a prostatic capsule, and the first portion generally corresponds to a neuro-vascular bundles and wherein the step of generating the ultrasound data includes the steps of positioning an ultrasound transducer proximate to the tissue treatment area by the transrectal insertion of the ultrasound transducer and obtaining multiple two-dimensional images of the tissue treatment area including a plurality of sector images and a plurality of linear images.

5. The method of claim 1, wherein substantially all of an interior of the first tissue component is treated with HIFU Therapy in the proposed treatment plan.

6. The method of claim 1, wherein a first portion of tissue in the tissue treatment area is excluded from the proposed treatment plan, the first portion of tissue generally corresponding to the location of a second tissue component.

7. The method of claim 6, wherein the location of the second tissue component is based on a three-dimensional model of the second tissue component, the three-dimensional model of the second tissue component being based on the generated ultrasound data.

8. The method of claim 7, wherein the first tissue component is a prostatic capsule and the second tissue component is selected from one of a rectal wall, a seminal vesicle, and an urethra.

9. The method of claim 1, wherein the three-dimensional model is generated by the steps of:
locating a first boundary trace of the first tissue component in a first set of ultrasound data generally corresponding to a first plane;
locating a second boundary trace of the first tissue component in a second set of ultrasound data generally corresponding to a second plane, the second plane being generally orthogonal to the first plane; and
computing a boundary surface of the first tissue component based on the first boundary trace and the second boundary trace.

10. The method of claim 1, further comprising the steps of:
presenting the proposed treatment plan on a display device along with a three dimensional representation of the tissue treatment area for review by a user;
determining a first location within the tissue treatment area having blood flow associated therewith;
further presenting on the display for review by a user an indicia to indicate the presence of blood flow at the first location, the indicia being positioned to correspond to the first location;
receiving a modification to the proposed treatment plan from the user thereby generating a modified proposed treatment plan; and
commencing the modified proposed treatment.

11. The method of claim 10, wherein the indicia provides an indication of the level of blood flow at the first location.

12. A method of providing treatment to a tissue treatment area including a plurality of tissue components, the method comprising the steps of:
generating ultrasound data related to the tissue treatment area;
generating blood flow data related to the tissue treatment area;
determining the location of a first tissue component based on the ultrasound data;
determining the location of a second tissue component based on the blood flow data; and
automatically generating a proposed treatment plan of the tissue treatment area, the proposed treatment plan including a plurality of treatment sites, the plurality of treatment sites being selected such that HIFU Therapy is provided to the first tissue component and such that the second tissue component is excluded from HIFU Therapy.

13. The method of claim 12, wherein the blood flow data is generated by Doppler ultrasound imaging and wherein the location of the first tissue component is determined based on a three-dimensional model of the first tissue component, the three-dimensional model of the first tissue component being based on the ultrasound data.

14. The method of claim 13, wherein the location of the second tissue component is determined based on an indication of the presence of blood flow at the location of the second tissue component and the relative position of the of the location of the second tissue component and the three-dimensional model of the first tissue component.

15. The method of claim 14, further comprising the steps of:
presenting on a display device for review by a user
a three dimensional representation of the tissue treatment area;
a plurality of treatment indicia, each of the treatment indicia corresponding to a respective treatment site,
a representation of a boundary of the first tissue component, the boundary being determined from the three-dimensional model of the first tissue component; and
a blood flow indicia indicating the location of the second tissue component, the blood flow indicia providing an indication of the amount of blood flow;
receiving a modification to the proposed treatment plan from the user thereby generating a modified proposed treatment plan; and
commencing the modified proposed treatment plan.

16. The method of claim 12, wherein the tissue treatment area generally corresponds to a prostate of a patient, the first tissue component generally corresponds to a prostatic capsule, and the second tissue component generally corresponds to a neuro-vascular bundles and wherein the step of generating the ultrasound data includes the steps of positioning an ultrasound transducer proximate to the tissue treatment area by the transrectal insertion of the transducer and obtaining multiple two-dimensional images of the tissue treatment area including a plurality of sector images and a plurality of linear images.

17. An apparatus for treating a tissue treatment area including a plurality of tissue components, the apparatus comprising:
a transducer which is positionable proximate to the tissue treatment area of tissue, the transducer being configured to emit ultrasound energy and to sense ultrasound energy; and
a controller operably coupled to the transducer, the controller being configured to operate the transducer in an imaging mode wherein images of the tissue are obtained by ultrasound energy sensed by the transducer and blood flow information and in a therapy mode wherein portions of the tissue in the tissue treatment area are treated with a HIFU Therapy with the transducer; the controller being further configured to automatically generate a proposed treatment plan having a plurality of treatment sites, the plurality of treatment sites of the proposed treatment plan being selected to provide HIFU Therapy to a first tissue component, while excluding a second tissue component from HIFU Therapy, the location of the second tissue component being determined based on blood flow information obtained during the imaging mode of operation.

18. The apparatus of claim 17, wherein the tissue treatment area generally corresponds to a prostate of a patient and wherein the transducer is contained within a probe, the probe being configured for transrectal insertion to position the transducer proximate to the tissue treatment area.

19. The apparatus of claim 18, wherein the first tissue component generally corresponds to a prostatic capsule, and the second tissue component generally corresponds to a neuro-vascular bundles.

20. The apparatus of claim 18, wherein the location of the first tissue component is determined based on a three-dimensional model of the first tissue component, the three-dimensional model being generated by the steps of:
locating a first boundary trace of the first tissue component in a first image obtained during the imaging mode of operation, the first image generally corresponding to a first plane;
locating a second boundary trace of the first tissue component in a second image obtained during the imaging mode of operation, the second image generally corresponding to a second plane, the second plane being generally orthogonal to the first plane; and
computing a boundary surface of the first tissue component based on the first boundary trace and the second boundary trace.

21. The apparatus of claim 20, wherein the boundary surface is based on Fourier ellipsoids.

22. The apparatus of claim 17, further comprising a display device, the controller being configured to present with the display device for review by a user a three dimensional representation of the tissue treatment area, a plurality of treatment indicia, each of the treatment indicia corresponding to a respective treatment site in the proposed treatment plan, a representation of a boundary of the first tissue component, the boundary being determined from a three-dimensional model of the first tissue component; and a blood flow indicia indicating the location of the second tissue component.

23. The apparatus of claim 22, wherein the blood flow indicia provides an indication of the amount of blood flow.

24. The apparatus of claim 22, further comprising an user input device, the controller being configured to generate a modified proposed treatment plan based on a requested modification received with the user input device.

25. The apparatus of claim 17, wherein the controller includes a multiple gate system for sampling Doppler information which provides the blood flow information, each gate of the multiple gate system being configured to sample a given time period of Doppler information.

26. An apparatus for treating a tissue treatment area including a plurality of tissue components, the apparatus comprising:
a transducer which is positionable proximate to the tissue treatment area, the transducer being configured to emit ultrasound energy and to sense ultrasound energy; and
a controller operably coupled to the transducer, the controller being configured to operate the transducer in an imaging mode wherein images of the tissue are obtained by ultrasound energy sensed by the transducer and in a therapy mode wherein portions of the tissue in the tissue treatment area of tissue are treated with a HIFU therapy with the transducer; the controller being further configured to automatically generate a proposed treatment plan of the tissue treatment area, the proposed treatment plan including a plurality of treatment sites selected based on a three-dimensional model of a first tissue component located in the tissue treatment area of tissue, the three-dimensional model of the first tissue component being based on the images obtained in the imaging mode of operation.

27. The apparatus of claim 26, wherein the tissue treatment area generally corresponds to a prostate of a patient and wherein the transducer is contained within a probe, the probe being configured for transrectal insertion to position the transducer proximate to the tissue treatment area.

28. The apparatus of claim 27, wherein the first tissue component is a prostatic capsule.

29. The apparatus of claim 26, wherein the three-dimensional model of the first tissue component is generated by the steps of:
locating a first boundary trace of the first tissue component in a first image obtained during the imaging mode of operation, the first image generally corresponding to a first plane;
locating a second boundary trace of the first tissue component in a second image obtained during the imaging mode of operation, the second image generally corresponding to a second plane, the second plane being generally orthogonal to the first plane; and
computing a boundary surface of the first tissue component based on the first boundary trace and the second boundary trace.

30. The apparatus of claim 26, further comprising a display device, the controller being configured to detect the presence of blood flow in the tissue treatment area of tissue and present with the display device for review by a user a three dimensional representation of the tissue treatment area including the three-dimensional model of the first tissue component, a plurality of treatment indicia, each of the treatment indicia corresponding to a respective treatment site in the proposed treatment plan, and a blood flow indicia indicating the location of blood flow in the tissue treatment area.

31. The apparatus of claim 30, wherein the blood flow indicia provides an indication of an amount of blood flow.

32. The apparatus of claim 26, further comprising an user input device, the controller being configured generate a modified proposed treatment plan based on a requested modification received with the user input device.

33. A method for treating tissue in a tissue treatment area including tissue components, comprising the steps of:
providing a HIFU system having software configured to provide therapy to diseased tissue by focusing ultrasound proximate to the diseased tissue and further configured to provide location information of blood flow in the tissue and location information on at least one of the tissue components;
identifying potential treatment areas based on the location information of the tissue components;
excluding potential treatment areas based on the location information of blood flow in the tissue treatment area; and
providing therapy to all of the identified non-excluded treatment areas with the HIFU system.

34. The method of claim 33, wherein the location information of the tissue components is presented to the a user by the following steps:
identifying the location of blood flow with Doppler imaging;
identifying the location of at least some of the tissue components with at least one of ultrasound 2-D imaging and ultrasound 3-D imaging;
generating a three-dimensional model representation of the location of tissue components based on the location of blood flow and the ultrasound imaging information; and
displaying the three-dimensional model on a display.

35. The method of claim 34, further comprising the step of overlaying representations of the treatment zones on the three-dimensional model.

36. The method of claim 35, further comprising the step providing a user input device associated with the HIFU system to permit the selection and deletion of the representations of the treatment zones.

37. The method of claim 36, wherein the deletion of the representation of the treatment site indicates to the HIFU system to exclude the area of tissue corresponding to the treatment site from therapy.

38. A method of providing treatment to a tissue treatment area including a prostatic capsule and a neuro-vascular bundles, the method comprising the steps of:
imaging the tissue treatment area; and
automatically generating a proposed treatment plan of the tissue treatment area, the proposed treatment plan including a plurality of treatment sites selected to receive HIFU Therapy, the plurality of treatment sites being selected to provide HIFU Therapy to the prostatic capsule and to exclude the neuro-vascular bundles from the provision of HIFU Therapy.

39. The method of claim 38, wherein the location of the neuro-vascular bundles is determined based on blood flow information obtained during the imaging of the tissue treatment area.

40. The method of claim 38, further comprising the steps of:
presenting the proposed treatment plan to a user for review;
receiving a modification to the proposed treatment plan from the user; and
generating a modified proposed treatment plan based on the received modification.
